(19) United States
(12) Patent Application Publication
(10) Pub. No.: US 2026/0026758 A1
(43) Pub. Date: Jan. 29, 2026

(54) SYSTEMS AND METHODS FOR TITRATING A BASAL INSULIN DOSE AND FOR DETECTING NON-COMPLIANCE WITH DOSE RECOMMENDATIONS

(71) Applicant: Abbott Diabetes Care Inc., Alameda, CA (US)

(72) Inventors: Aparajita BHATTACHARYA, Dublin, CA (US); Gary A. HAYTER, Oakland, CA (US); Byron Paul OLSON, II, Sioux Falls, SD (US); Matthew James CLEMENTE, Milpitas, CA (US)

(21) Appl. No.: 19/281,136

(22) Filed: Jul. 25, 2025

Related U.S. Application Data
(60) Provisional application No. 63/676,205, filed on Jul. 26, 2024.

(51) Int. Cl. A61B 5/00; A61B 5/145

(52) U.S. Cl. CPC A61B 5/7282; A61B 5/14532; A61B 5/4833; A61B 5/742; A61B 5/7475

(57) ABSTRACT

A system for titrating a basal insulin dose that includes a glucose monitoring device configured to collect glucose data of a user, processors in communication with the glucose monitoring device, and a memory coupled to the processors. The processors are configured to determine an initial basal insulin dose, receive insulin data and glucose data during a titration period. The validity of each day is assessed, and a titration glucose level is determined for each valid day. The titration glucose level for each day is compared to glucose level thresholds, and a recommended adjustment to the basal insulin dose is determined based on the comparison.

### CROSS-REFERENCE TO RELATED APPLICATION

This application claims priority to and the benefit of U.S. Provisional Application No. 63/676,205, filed Jul. 26, 2024 which is incorporated herein by reference in its entirety.

### FIELD OF THE INVENTION

Some embodiments described herein relate to computer-implemented systems and methods for titrating a medication dose. Some embodiments relate to titrating a basal insulin dose based on glucose data collected by a glucose monitoring device. Some embodiments relate to detecting user non-compliance or compliance with dose recommendations output by a dose guidance system.

### BACKGROUND

Patients with diabetes must carefully monitor their glucose levels to ensure their glucose levels remain in a target range. If glucose levels are too high, the user may experience negative health complications of hyperglycemia. If glucose levels are too low, the user may experience negative health complications of hypoglycemia. Patients may work with their healthcare provider to modify their diet and exercise to help to maintain glucose levels. If such methods are not effective in achieving glucose control, the patient may begin taking one or more medications to help control glucose levels.

Determining a therapy regimen to help the user to achieve and optimize glucose control can be difficult. There are various medications that are available for diabetic patients, and determining the proper combination of medications and doses for each medication can be challenging for healthcare professionals. Further, the therapy regimen is unique for each patient, and an approach that works for one patient may not work for another. The patient's health and lifestyle may change over time and the appropriate therapy regimen may also need to be revised. Accordingly, determining a therapy regimen for a patient can be difficult and may require trial and error over an extended period of time.

### SUMMARY OF THE INVENTION

Aspects of the invention are set out in the independent claims and preferred features are set out in the dependent claims. Features associated with one aspect may be applied to other aspects alone or in combination. Some embodiments described herein relate to a system for titrating a basal insulin dose that includes a glucose monitoring device configured to collect glucose data of a patient, one or more processors in communication with the glucose monitoring device, and a memory coupled to the one or more processors and storing instructions. When the instructions are executed by the one or more processors, the one or more processors are caused to: determine an initial basal insulin dose, receive insulin data during (or for) a titration period, wherein the titration period comprises a plurality of days, and receive glucose data for each day of the plurality of days in the titration period. In some embodiments, the system may not include the glucose monitoring device. For example, the one or more processors and/or memory may have access to glucose data collected by a glucose monitoring device (which may be external to the system or not in direct communication with the system). Further, the processors may be caused to assess a validity of each of the plurality of days in the titration period. Assessing the validity of each of the plurality of days may include assessing a validity of the insulin data and/or the glucose data for each of the plurality of days, and/or a validity of a length of each of the plurality of days. Further, the processors are caused to determine a titration glucose level for each day in the titration period that is determined to be valid, compare the titration glucose level for each day in the titration period that is determined to be valid to one or more glucose level thresholds, and recommend an adjustment to the basal insulin dose based on the comparison. In some embodiments, the processors may be caused to determine a titration glucose level for each day in the titration period (without assessing a validity of the plurality of days) and compare the titration glucose level for each day in the titration period to one or more glucose level thresholds, and output a recommended adjustment to the basal insulin dose based on the comparison.

In any of the various embodiments described herein, the titration glucose level may be determined by dividing each day into a plurality of time blocks, and calculating an average glucose level for each of the plurality of time blocks, wherein the titration glucose level is a lowest average glucose level of the plurality of time blocks.

The titration period may be divided or segmented into the plurality of days (time periods). Each of the plurality of days or time periods may be defined as a time period between consecutive basal insulin doses. In any of the various embodiments described herein, each day of the plurality of days may be based on a time period between a first basal insulin dose and a second basal insulin dose. In some embodiments, the day may be assessed to be valid when the time period is greater than or equal to a minimum duration. In some embodiments, the day may be assessed to be invalid when the time period is less than the minimum duration.

In some embodiments, the day may be assessed to be valid when the time period is less than or equal to a maximum duration.

In any of the various embodiments described herein, the recommended adjustment may be to decrease the basal insulin dose when the titration glucose level is below a lower glucose level threshold of the one or more glucose level thresholds meets or exceeds a minimum down-titration period threshold.

In any of the various embodiments described herein, the recommended adjustment may be to increase the basal insulin dose by a first amount when a number of days in which the titration glucose level meets or exceeds a first upper glucose level threshold of the one or more glucose level thresholds meets or exceeds a minimum up-titration period threshold. In some embodiments, the recommended adjustment may be to increase the basal insulin dose by a second amount when a number of days in which the titration glucose level meets or exceeds a second upper glucose level threshold of the one or more glucose level thresholds meets or exceeds the minimum up-titration period threshold, wherein the second upper glucose level threshold is greater than the first upper glucose level threshold, and wherein the second amount is greater than the first amount.

In any of the various embodiments described herein, the recommended adjustment to the basal insulin dose may be output at an end of the titration period and without user input.

In any of the various embodiments described herein, the initial basal insulin dose may be determined based on a body weight of the user.

In any of the various embodiments described herein, the one or more processors may further be caused to detect a low glucose event in a period of time following administration of a basal insulin dose, and the recommended adjustment is to decrease the basal insulin dose based on detection of the low glucose event. In some embodiments, the low glucose event may include glucose levels of the user being below a low glucose level threshold for at least a minimum period of time.

In any of the various embodiments described herein, the one or more processors may be configured to determine overbasalization when a ratio of the basal insulin dose to a body weight of the user meets or exceeds a dose to body weight ratio threshold, and when overbasalization is determined, the one or more processors may be configured to stop output of recommendations to increase the basal insulin dose.

In any of the various embodiments described herein, at least one of the one or more glucose level thresholds may be adjusted based on a sensor bias of the glucose monitoring device.

Some embodiments described herein relate to a method for titrating a basal insulin dose, wherein the method includes determining an initial basal insulin dose, receiving insulin data during (or for) a titration period, wherein the titration period comprises a plurality of days, and receiving glucose data for each day of the plurality of days in the titration period, wherein the glucose data is received from a glucose monitoring device comprising sensor electronics coupled to an in vivo glucose sensor. The method further includes assessing a validity of each of the plurality of days in the titration period. Assessing the validity of each of the plurality of days may include assessing a validity of the insulin data and/or the glucose data for each of the plurality of days, and/or a validity of a length of each of the plurality of days. The method further includes determining a titration glucose level for each day in the titration period that is determined to be valid, comparing the titration glucose level for each day in the titration period that is determined to be valid to one or more glucose level thresholds, and recommending an adjustment to the basal insulin dose based on the comparison. In some embodiments, the method may include determining a titration glucose level for each day in the titration period (without assessing a validity of the plurality of days) and comparing the titration glucose level for each day in the titration period to one or more glucose level thresholds, and recommending an adjustment to the basal insulin dose based on the comparison.

In any of the various embodiments described herein, the method may further include determining the titration glucose level by dividing each day into a plurality of time blocks, and calculating an average glucose level for each of the plurality of time blocks, wherein the titration glucose level is a lowest average glucose level of the plurality of time blocks.

In any of the various embodiments described herein, assessing the validity of each of the plurality of days may be based on a comparison of a duration of each day to a minimum duration and a maximum duration.

In any of the various embodiments described herein, the recommended adjustment may be to decrease the basal insulin dose when a number of days in which the titration glucose level is below a lower glucose level threshold of the one or more glucose level threshold meets or exceeds a minimum down-titration period threshold.

In any of the various embodiments described herein, the recommendation may be to increase the basal insulin dose by a first amount when a number of days in which the titration glucose level meets or exceeds an upper glucose threshold of the one or more glucose level thresholds meets or exceeds a minimum up-titration period threshold.

In any of the various embodiments described herein, the method may further include detecting a low glucose event in a period of time following administration of a basal insulin dose, and recommending an adjustment to decrease the basal insulin dose based on detection of the low glucose event.

In any of the various embodiments described herein, the method may further include detecting overbasalization when a ratio of the basal insulin dose to a body weight of the user meets or exceeds a dose to body weight ratio threshold, and when overbasalization is detected, stopping output of recommendations to increase the basal insulin dose.

### BRIEF DESCRIPTION OF THE FIGURES/DRAWINGS

The accompanying drawings, which are incorporated herein and form a part of the specification, illustrate the present disclosure and, together with the description, further serve to explain the principles thereof and to enable a person skilled in the pertinent art to make and use the same.

FIG. 1 shows an exemplary dose guidance system according to an embodiment.

FIG. 2 shows an exemplary dose guidance system according to an embodiment.

FIG. 3 shows a schematic diagram depicting an analyte monitoring device according to an embodiment.

FIG. 4 shows a block diagram of an analyte monitoring device according to an embodiment.

FIG. 5 shows a schematic diagram of a display device according to an embodiment.

FIG. 6 shows a block diagram depicting a display device according to an embodiment.

FIG. 7 shows a method for titrating a basal insulin dose according to an embodiment.

FIG. 8 shows a method for assessing validity of a day of data for use in a titration determination according to an embodiment.

FIG. 9 shows a method for assessing validity of a day of data for use in a titration determination according to another embodiment.

FIG. 10 shows a graph of glucose levels over a day illustrating a time block for determining a titration glucose level according to an embodiment.

FIG. 11 shows a method for titrating a basal insulin dose according to an embodiment.

FIG. 12 shows a method for titrating a basal insulin dose accounting for low glucose events according to an embodiment.

FIG. 13 shows an exemplary method of determining compliance with dose recommendations based on user interaction with the dose guidance system.

FIG. 14 shows an exemplary method of determining compliance with dose recommendations based on user interaction with the dose guidance system.

FIG. 15 shows an interface for prompting a user to confirm a new dose recommendation according to an embodiment.

FIG. 16 shows an exemplary method for initiating a corrective action when the user is determined to be non-compliant according to an embodiment.

FIG. 17 shows an exemplary method for determining compliance with dose recommendations based on collected glucose data.

FIG. 18 shows an exemplary plot illustrating a relationship of TGL and dose amount.

FIG. 19 shows an exemplary plot of TGL and dose amounts illustrating the slopes for a current TGL-dose pair relative to the TGL for all other dose amounts.

FIG. 20 shows an exemplary plot of TGL for each administered dose to illustrate TGL median and variability.

FIG. 21 shows an exemplary method for determining compliance with dose recommendations based on glucose data and accounting for risk of sudden compliance.

FIG. 22 shows an exemplary method of determining compliance with a dose recommendation based on an expected glucose metric.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein comport with standards used by those experienced or skilled in the art to most effectively convey the substance of their work to others skilled in the art. In some instances, well-known methods, procedures, and components have not been described in detail to avoid unnecessarily obscuring aspects of the disclosure.

References in the specification to “some embodiments” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to apply such feature, structure, or characteristic in connection with other embodiments whether or not explicitly described.

The following examples are illustrative, but not limiting, of the present disclosure. Other suitable modifications and adaptations of the variety of conditions and parameters normally encountered in the field, and which would be apparent to those skilled in the art, are within the spirit and scope of the disclosure.

Insulin may be initiated in the course of treating a patient with diabetes, and the dose of insulin may be titrated over time to help the patient to achieve proper glucose control. Different types of insulin may be used, such as basal insulin, also referred to as long-acting insulin. Basal insulin is typically administered once per day. Basal insulin may be taken alone or in combination with other therapies. Another type of insulin is rapid-acting insulin, also referred to as meal-time insulin. Rapid-acting insulin is often taken at the time of a meal, or just before one or more meals in a day, such as at one or more of breakfast, lunch, or dinner. Other types of insulin include intermediate-acting insulin and premixed insulin, which is a combination of insulin types, such as a combination of intermediate and rapid-acting insulin.

The American Diabetes Association (ADA) provides general guidelines for basal insulin initiation and titration. Specifically, the ADA recommends initiating basal insulin at 10 units/day or 0.1 to 0.2 units per kg of body weight per day. The ADA recommends titrating the basal insulin dose using an evidence-based titration algorithm, such as to increase the dose by 2 units every 3 days to reach a fasting plasma glucose (FPG) target level without hypoglycemia.

The ADA guidelines leave considerable room for interpretation in terms of selecting an initial insulin dose, the amount of adjustment of the dose when titrating, the titration frequency, and glucose targets. Further, the ADA guidelines provide no indication of how to use continuous glucose monitor (CGM) data or whether or how to consider information regarding patient compliance with insulin therapy. The present application aims to address the deficiencies in the prior art and to implement a basal insulin titration algorithm in a dose guidance system including a glucose monitoring device.

The healthcare provider (HCP) may review a patient's insulin and glucose data in an appointment with the patient and may manually determine whether and how to adjust the basal insulin dose. The HCP may rely on the patient's reporting of insulin doses, and one or more blood glucose measurements to indicate a FPG level. This process of manual titration by the HCP has considerable drawbacks and deficiencies. The patient may only visit a HCP once every few months, or just a few times per year. As a result, the titration of the basal insulin dose may occur very gradually, and the patient may suffer from poor glucose control in the interim. FPG levels used to determine the effectiveness of the basal insulin dose in controlling glucose levels may be determined based on a small number of blood glucose measurements which may not be representative of the user's glucose control over time. Further, the HCP may have little information to determine if the patient is complying with the prescribed therapy regimen and is routinely administering the prescribed basal insulin dose at appropriate times. Patients may keep poor records of their medication history, may intentionally or accidentally fail to take doses of medication, may take the wrong dose amount, or may be unwilling to admit to failing to comply with the therapy. As a result, the HCP may adjust medication doses where the patient is not complying with therapy. Additionally, even when indicated, the HCP may be reluctant to increase the dose due to the complexity of the titration determination to avoid increasing incidence of hypoglycemia.

To address the issue of limited glucose data, continuous glucose monitors are available that facilitate collection of glucose data on a continuous basis. The continuous glucose data may provide a better picture of the user's overall glucose control. Using the continuous glucose data may allow for better understanding of the patient's glucose levels, and may allow for a more precise adjustments to be made to the user's basal insulin dose. However, interpreting the voluminous continuous glucose data to determine how to adjust insulin therapy can be difficult, particularly in a short period in which the HCP visits with the patient. Further, as discussed, the ADA guidelines provide no explanation for how to utilize or interpret continuous glucose data in titrating the basal insulin dose.

Accordingly, there is a need in the art for a dose guidance system that is compliant with ADA guidelines and that facilitates collection and analysis of glucose and insulin data to improve titration of basal insulin doses. Further, there is a need in the art for a dose guidance system that receives insulin data and glucose data from connected devices, such as a continuous glucose monitor and smart insulin pen or pen cap, and determines titration recommendations to allow for more frequent titration of the basal insulin dose than is provided by relatively infrequent HCP visits. Automatically receiving data from connected devices also helps to facilitate data collection and to ensure the accuracy of data. The dose guidance system incorporating glucose data would provide significantly more data on which to make titration determinations. A fasting glucose value may be more accurately determined based on continuous glucose data and eliminates the need for fingerstick blood glucose measurements. Further, the user's compliance with the therapy may be detected, and as a result titration decisions may be made based on more reliable information.

Dose guidance systems for titrating a basal insulin dose are described herein. For simplicity, a “dose guidance system” as described herein may be referred to simply as the “system.” The dose guidance system or “system” may include algorithms or software for titrating a basal insulin dose and for performing the methods as described herein. The algorithms may include instructions stored in memory and executed by one or more processors coupled to or in communication with the memory. The algorithms may be executed on one or more processors of a display device of a user, such as a mobile device, e.g., a smartphone, a dedicated handheld display device associated with an analyte sensor, among other portable electronic devices, and other computing devices. The algorithms may be included in a mobile application with a graphical user interface on a display of the display device. The algorithms may be executed at a remote computer or server, and may communicate with the display device to provide medication information, such as dose recommendations. The algorithms may be executed on a medication delivery device, such as a pump or injection pen or associated controller or display, or dose monitoring device, such as a smart pen cap, among others. The algorithms may be executed by a combination of a display device, medication delivery device, and remote computer or server.

The dose guidance system may include one or more algorithms as described herein. For example, a titration algorithm may be based on ADA guidelines (e.g., may implement the ADA standards or guidelines). Another titration algorithm may be personalized to the user, and may include user-defined values or values that are dynamically determined over time based on the user's glucose and insulin data. A titration algorithm may be based on machine learning or artificial intelligence. The user, such as a patient or HCP, may select a titration algorithm for use by the patient. The selection may be based on the patient's goals, such as for conservative or aggressive treatment. The dose guidance system may begin with a first algorithm and transition to a second algorithm over time. For example, dose guidance system may start therapy with a conservative titration algorithm, and may switch to a personalized titration algorithm once the user has used the dose guidance system for a period of time, and sufficient user data has been collected.

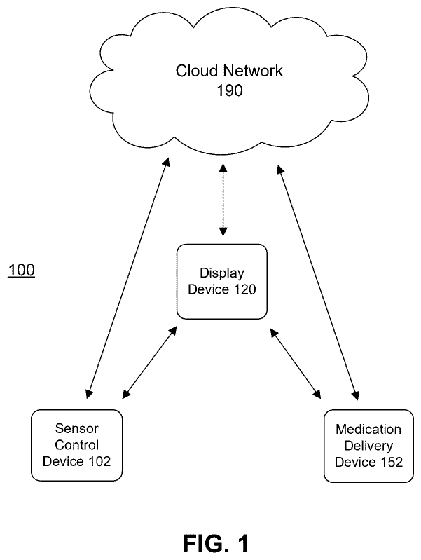

FIG. 1 is a block diagram depicting an example embodiment of dose guidance system 100. In this embodiment, dose guidance system 100 is capable of providing dose guidance, monitoring one or more analytes, and delivering one or more medications. This multifunctional example is used to illustrate the high degree of interconnectivity and performance obtainable by dose guidance system 100. However, in the embodiments described herein, the analyte monitoring components, the medication delivery components, or both can be omitted if desired.

System 100 may include one or more of a sensor control device 102 configured to collect analyte level information from a user, a medication delivery device 152 configured to deliver medication to the user, and a display device 120 configured to present information to the user and receive input or information from the user. The structure and function of each device will be described in further detail herein.

System 100 is configured for highly interconnected and highly flexible communication between devices. Each of the three devices 102, 120, and 152, can communicate directly with each other (without passing through an intermediate electronic device) or indirectly with each other (such as through cloud network 190, or through another device and then through cloud network 190). Bidirectional communication capability between devices, as well as between devices and network 190, is shown in FIG. 1 with a double-sided arrow. However, those of skill in the art will appreciate that any of the one or more devices can be capable of unidirectional communication such as, for example, broadcasting, multicasting, or advertising communications. In each instance, whether bidirectional or unidirectional, the communication can be wired or wireless. The protocols that govern communication over each path can be the same or different, and can be either proprietary or standardized. For example, wireless communication between devices 102, 120, and 152 can be performed according to a Bluetooth (including Bluetooth Low Energy) standard, a Near Field Communication (NFC) standard, a Wi-Fi (802.11x) standard, a mobile telephony standard, or others. All communications over the various paths can be encrypted, and each device of FIG. 1 can be configured to encrypt and decrypt those communications sent and received. In each instance the communication pathways of FIG. 1 can be direct (e.g., Bluetooth or NFC) or indirect (e.g., Wi-Fi, mobile telephony, or other internet protocol). Embodiments of system 100 do not need to have the capability to communicate across all of the pathways indicated in FIG. 1.

The analyte monitoring functionality of dose guidance system 100 can be realized through inclusion of one or more devices capable of collecting, processing, and displaying analyte data of the user. Example embodiments of such devices and their methods of use are described in International Publ. No. WO 2018/152241 and U.S. Pat. No. 10,136,816 B2, both of which are incorporated by reference herein in their entireties for all purposes.

Analyte monitoring can be performed in numerous different ways. “Continuous Analyte Monitoring” devices (e.g., “Continuous Glucose Monitoring” devices), for example, can transmit data from a sensor control device to a display device continuously or repeatedly with or without prompting, e.g., automatically according to a schedule. “Flash Analyte Monitoring” devices (e.g., “Flash Glucose Monitoring” devices or simply “Flash” devices), as another example, can transfer data from a sensor control device in response to a user-initiated request for data by a display device (e.g., a scan), such as with a Near Field Communication (NFC) or Radio Frequency Identification (RFID) protocol.

Analyte monitoring devices that utilize a sensor configured to be placed partially or wholly within a user's body can be referred to as in vivo analyte monitoring devices. The in vivo analyte monitoring device may be used to measure glucose levels and may be referred to as an in vivo glucose monitoring device. For example, an in vivo analyte sensor can be placed in the user's body such that at least a portion of the analyte sensor is in contact with a bodily fluid (e.g., interstitial (ISF) fluid such as dermal fluid in the dermal layer or subcutaneous fluid beneath the dermal layer, blood, or others) and can measure an analyte concentration in that bodily fluid. In vivo analyte sensors can use various types of sensing techniques (e.g., chemical, electrochemical, or optical). Some systems utilizing in vivo analyte sensors can also operate without the need for finger stick calibration.

In some embodiments, the glucose monitoring device includes an in vivo glucose sensor coupled or coupleable to sensor electronics. The sensor electronics may be permanently coupled to the glucose sensor, or the sensor electronics may be configured to be coupled to the glucose sensor during insertion of the glucose sensor under the skin of a user, or by coupling the sensor electronics to the glucose sensor after the glucose sensor is implanted under the skin of the user. The glucose sensor includes a first portion configured to be positioned above a skin surface of a user and a second portion configured to be positioned below a skin surface and in contact with a bodily fluid of the user for monitoring glucose levels in the bodily fluid. The second portion may include one or more electrodes. In some embodiments, the electrodes may include a working electrode having one or more active areas. The active area may include sensing chemistry for detection of glucose in the bodily fluid. The sensing chemistry may include one or more enzymes, such as glucose oxidase, or glucose dehydrogenase, among others.

Sensor electronics are coupled to the first portion of the in vivo glucose sensor. The sensor electronics may include communication circuitry, such as one or more antennas, for transmitting glucose data by a wireless communication protocol, such as Bluetooth communication protocol or Near Field Communication (NFC) protocol, among others. Sensor electronics may further include one or more processors configured to process signals collected by the glucose sensor, such as to determine a glucose concentration value. Sensor electronics may further include a power source, such as one or more batteries.

The present subject matter will be described primarily with respect to glucose monitoring devices and glucose sensors capable of measuring a glucose concentration, although detection and measurement of concentrations of alternate or additional analytes are within the scope of the present disclosure. These other analytes can include, for example, ketones, lactate, or alcohol. Further analytes may include oxygen, hemoglobin A1C, acetyl choline, amylase, bilirubin, cholesterol, chorionic gonadotropin, creatine kinase (e.g., CK-MB), creatine, DNA, fructosamine, glutamine, growth hormones, hormones, peroxide, prostate-specific antigen, prothrombin, RNA, thyroid stimulating hormone, troponin and others. The concentration of drugs, such as, for example, antibiotics (e.g., gentamicin, vancomycin, and the like), digitoxin, digoxin, drugs of abuse, theophylline, and warfarin, may also be monitored. The analyte sensor can be configured to measure two or more different analytes at the same or different times. The sensor control device can be coupled with two or more sensors, where one analyte sensor is configured to measure a first analyte (e.g., glucose) and the other one or more analyte sensors are configured to measure one or more different analytes (e.g., any of those described herein). In other embodiments, a user can wear two or more sensor control devices, each of which is capable of measuring a different analyte.

The analyte sensor operation can be controlled by sensor control device 102. The analyte sensor can be mechanically and communicatively coupled with sensor control device 102, or can be just communicatively coupled with sensor control device 102 using a wireless communication technique. Sensor control device 102 can include the sensor electronics and power supply that enable and control analyte sensing performed by the sensor. The sensor or sensor control device 102 can be self-powered such that a battery is not required. Sensor control device 102 can also include communication circuitry for communicating with another device that may or may not be local to the user's body (e.g., a display device). Sensor control device 102 can reside on the body of the user (e.g., attached to or otherwise placed on the user's skin, or carried in the user's clothes, etc.). Sensor control device 102 can also be implanted within the body of the user along with the sensor. Functionality of sensor control device 102 can be divided between a first component implanted within the body (e.g., a component that controls the sensor) and a second component that resides on or otherwise outside the body (e.g., a relay component that communicates with the first component and also with an external device like a computer or smartphone). The sensor control device, depending on the actual implementation or embodiment, can also be referred to as a “sensor control unit,” an “on-body electronics” device or unit, an “on-body” device or unit, an “in body electronics” device or unit, an “in-body” device or unit, or a “sensor data communication” device or unit, to name a few.

Sensor control device 102 may include a user interface (e.g., a touchscreen) and be capable of processing the analyte data and displaying the resultant calculated analyte levels to the user. In such cases, the dose guidance embodiments described herein can be implemented directly by sensor control device 102, in whole or in part. In many embodiments, the physical form factor of sensor control device 102 is minimized (e.g., to minimize the appearance on the user's body) or sensor control device 102 may be inaccessible to the user (e.g., if wholly implanted), or other factors may make it desirable to have a display device usable by the user to read analyte levels and interface with the sensor control device.

Although FIG. 1 depicts a single display device 120, a single sensor control device 102, and a single medication delivery device 152, those of skill in the art will appreciate that system 100 can comprise a plurality of any of the aforementioned devices. By way of example only, system 100 can comprise a single sensor control device 102 in communication with multiple (e.g., two, three, four, etc.) display devices 120 and/or multiple medication delivery devices 152. Alternatively, system 100 can comprise a plurality of sensor control devices 102 in communication with a single display device 120 and/or a single medication delivery device 152. Furthermore, each of the plurality of devices can be of the same or different device types. For example, system 100 can comprise multiple display devices 120, including a smart phone, a handheld receiver, and/or a smart watch, each of which can be in communication with sensor control device 102 and/or medication delivery device 152, as well in communication with each other (e.g., via Bluetooth or other wireless communication methods). Any of sensor control device 102, display device 120, medication delivery device 152 or cloud network 190, or combinations thereof, may store and/or execute dose guidance algorithms as discussed herein.

Analyte data and other data can be transferred between each device within system 100 in an autonomous fashion (e.g., transmitting automatically according to a schedule), or in response to a request for analyte data (e.g., sending a request from a first device to a second device for analyte data, followed by transmission of the analyte data from the second device to the first device). Other techniques for communicating data can also be employed to accommodate more complex systems like cloud network 190.

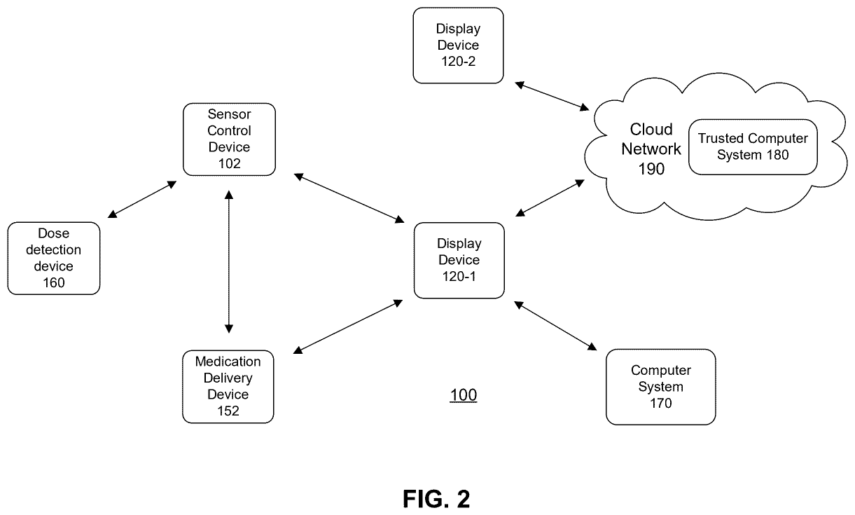

FIG. 2 is a block diagram depicting another example embodiment of dose guidance system 100. Here, system 100 includes one or more of a sensor control device 102, medication delivery device 152, a first dose detection device 160, a first display device 120-1, a second display device 120-2, local computer system 170, and trusted computer system 180 that is accessible by cloud network 190. Dose guidance algorithms as disclosed herein may be stored and/or executed by any of sensor control device 102, medication delivery device 152, first dose detection device 160, first display device 120-1, second display device 120-2, local computer system 170, trusted computer system 180, or cloud network 190, or combinations thereof. Sensor control device 102 and medication delivery device 152 are capable of communication with each other and with display device 120-1, which can act as a communication hub for aggregating information from sensor control device 102 and medication delivery device 152, processing and displaying that information where desired, and transferring some or all of the information to cloud network 190 and/or computer system 170. Conversely, display device 120-1 can receive information from cloud network 190 and/or computer system 170 and communicate some or all of the received information to sensor control device 102, medication delivery device 152, or both. Computer system 170 may be a personal computer, a server terminal, a laptop computer, a tablet, or other suitable data processing device. Computer system 170 can include or present software for data management and analysis and communication with the components in dose guidance system 100. Computer system 170 can be used by the user or a medical professional to display and/or analyze analyte data measured by sensor control device 102. Furthermore, although FIG. 2 depicts a single sensor control device 102, a single medication delivery device 152, a single dose detection device 160, and two display devices 120-1 and 120-2, those of skill in the art will appreciate that dose guidance system 100 can include a plurality of any of the aforementioned devices, wherein each plurality of devices can comprise the same or different types of devices.

Referring still to FIG. 2, according to some embodiments, trusted computer system 180 can be within the possession of a manufacturer or distributor of a component of dose guidance system 100, either physically or virtually through a secured connection, and can be used to perform authentication of the devices of system 100 (e.g., devices 102, 120-n, 152, 160), for secure storage of the user's data, and/or as a server that serves a data analytics program (e.g., accessible via a web browser) for performing analysis on the user's measured analyte data and medication history. Trusted computer system 180 can also act as a data hub for routing and exchanging data between all devices in communication with system 180 through cloud network 190. In other words, all devices of dose guidance system 100 that are capable of communicating with cloud network 190 (e.g., either directly with an internet connection or indirectly via another device), are also capable of communicating with all of the other devices of dose guidance system 100 that are capable of communicating with cloud network 190, either directly or indirectly.

Display device 120-2 is depicted in communication with cloud network 190. In this example, device 120-2 can be in the possession of another user that is granted access to the analyte and medication data of the person wearing sensor control device 102. For example, the person in possession of display device 120-2 can be a parent of a child wearing sensor control device 102, as one example, or a caregiver of an elderly patient wearing sensor control device 102, as another example. Dose guidance system 100 can be configured to communicate analyte and medication data about the wearer through cloud network 190 (e.g., via trusted computer system 180) to another user with granted access to the data.

Medication delivery device 152 may include an infusion pump, a patch pump, or an injection pen, among other devices for administering a medication. Dose guidance system 100 may include one or more dose detection devices 160 (see FIG. 2), such as a smart pen cap configured to be removably disposed on an injection pen to collect medication information, such as the type of medication, time of administration of a dose, or dose amount, among other information. Dose detection device 160 may directly detect administration of a dose, such as by tracking movement of one or more components of an injection device, such as a plunger, dose knob, dose window or markings therein, or the like. Dose detection device 160 may detect administration of a dose via any of various methods, such as by one or more sensors, such as an optical sensor, magnetic sensor, or via one or more switches, among other methods. Alternatively or additionally, dose detection device 160 may infer administration of a dose based on movement of dose detection device relative to the medication delivery device, such as by uncapping the pen cap from an injection pen, recapping the pen cap onto the injection pen, or a combination thereof. Dose detection device 160 may communicate with sensor control device 102 such as to receive analyte data. Dose detection device 160 may communicate with display devices 120, with cloud network 190 or trusted computer system 170 directly or indirectly via other components of dose guidance system 100. Dose detection device 160 may communicate dose information and may receive data from other components.

Dose guidance system 100 may determine a dose of medication to administer and may display the recommended dose on a display device 120 or dose detection device 160. The user may then use a medication delivery device 152 to administer the recommended dose. The medication delivery device 152 or dose detection device 160 may communicate collected dose information to dose guidance system 100. Dose information may include one or more of a time of a dose, an amount of a dose, a type of medication, or a time since a last dose, among other information. Alternatively, dose guidance system 100 may determine a dose of medication to administer and may communicate the recommended dose to medication delivery device 152. Medication delivery device 152 may display the recommended dose. Medication delivery device 152 may automatically administer the dose determined by dose guidance system 100 or may require user action to administer the dose.

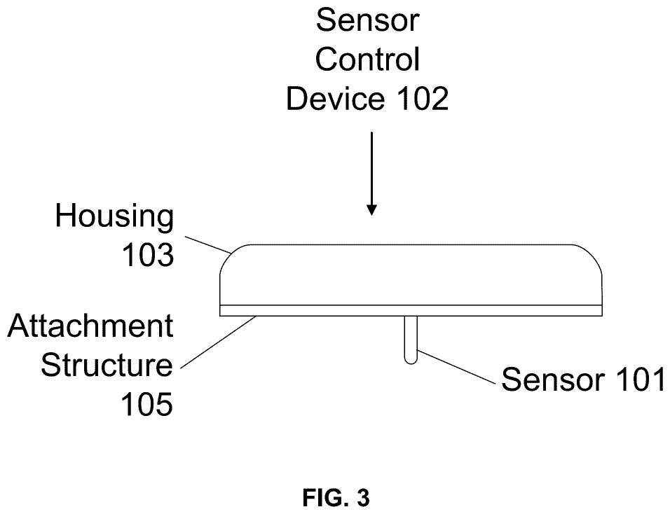

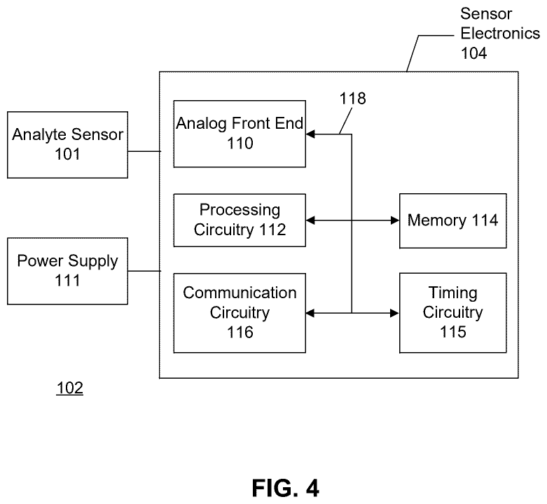

FIG. 3 is a side view of an example embodiment of sensor control device 102. Sensor control device 102 may be referred to as an analyte monitoring device, or as a glucose monitoring device where the analyte to be monitored is glucose (e.g., a CGM device). Sensor control device 102 can include a housing or mount 103 for sensor electronics (FIG. 4), which can be electrically coupled with an analyte sensor 101, which is configured here as an electrochemical sensor. According to some embodiments, sensor 101 can be configured to reside partially within a user's body (e.g., through an exterior-most surface of the skin) where it can make fluid contact with a user's bodily fluid and be used, along with the sensor electronics, to measure analyte-related data of the user. A portion of sensor configured to be arranged under the skin may be in the form of a thin filament or wire. The portion of the sensor arranged under the skin may be elongated, having a long length relative to diameter, and may be generally linear. An attachment structure 105, such as an adhesive patch, can be used to secure housing 103 to a user's skin. Sensor 101 can extend through attachment structure 105 and project away from housing 103. Those of skill in the art will appreciate that other forms of attachment to the body and/or housing 103 may be used, in addition to or instead of adhesive, and are fully within the scope of the present disclosure.

Sensor 101 may include one or more electrodes. Electrodes may include a working electrode and a counter electrode and/or reference electrode. In some embodiments, sensor 101 may include multiple working electrodes. Electrodes may be arranged on a substrate, such as a planar substrate. Electrodes may be arranged on one or both faces of the substrate. Electrodes may be separated by dielectric layers. A sensing element may be arranged on one or more of the electrodes, such as on the working electrode. The sensing element may be configured to facilitate detection of an analyte of interest, such as glucose, among others. The sensing element may include an analyte-responsive enzyme. One or more membranes may at least partially cover sensor 101 for biocompatibility and diffusion control, or a combination thereof. The electrodes may be in electrical connection with one or more contacts arranged on another portion of sensor 101 that is in communication with sensor electronics. Electrodes may be connected to electrical contacts by electrical traces that extend along sensor 101.

Sensor control device 102 can be applied to the body in any desired manner. For example, an insertion device (not shown), sometimes referred to as an applicator, can be used to position all or a portion of analyte sensor 101 through an external surface of the user's skin and into contact with the user's bodily fluid. In doing so, the insertion device can also position sensor control device 102 onto the skin. In other embodiments, the insertion device can position sensor 101 first, and then accompanying electronics (e.g., wireless transmission circuitry and/or data processing circuitry, and the like) can be coupled with sensor 101 afterwards (e.g., inserted into a mount), either manually or with the aid of a mechanical device. Examples of insertion devices are described in U.S. Patent Publication Nos. 2008/0009692, 2011/0319729, 2015/0018639, 2015/0025345, and 2015/0173661, 2018/0235520, all which are incorporated by reference herein in their entireties for all purposes.

FIG. 4 is a block diagram depicting an example embodiment of sensor control device 102 having analyte sensor 101 and sensor electronics 104. Sensor electronics 104 can be implemented in one or more semiconductor chips (e.g., an application specific integrated circuit (ASIC), processor or controller, memory, programmable gate array, and others). In the embodiment of FIG. 4, sensor electronics 104 includes high-level functional units, including an analog front end (AFE) 110 configured to interface in an analog manner with sensor 101 and convert analog signals to and/or from digital form (e.g., with an A/D converter), a power supply 111 configured to supply power to the components of sensor control device 102, processing circuitry 112, memory 114, timing circuitry 115 (e.g., such as an oscillator and phase locked loop for providing a clock or other timing to components of sensor control device 102), and communication circuitry 116 configured to communicate in wired and/or wireless fashion with one or more devices external to sensor control device 102, such as display device 120 and/or medication delivery device 152.

Sensor control device 102 can be implemented in a highly interconnected fashion, where power supply 111 is coupled with each component shown in FIG. 4 and where those components that communicate or receive data, information, or commands (e.g., AFE 110, processing circuitry 112, memory 114, timing circuitry 115, and communication circuitry 116), can be communicatively coupled with every other such component over, for example, one or more communication connections or buses 118.

Processing circuitry 112 can include one or more processors, microprocessors, controllers, and/or microcontrollers, each of which can be a discrete chip or distributed amongst (and a portion of) a number of different chips. Processing circuitry 112 can include on-board memory. Processing circuitry 112 can interface with communication circuitry 116 and perform analog-to-digital conversions, encoding and decoding, digital signal processing and other functions that facilitate the conversion of data signals into a format (e.g., in-phase and quadrature) suitable for wireless or wired transmission. Processing circuitry 112 can also interface with communication circuitry 116 to perform the reverse functions necessary to receive a wireless transmission and convert it into digital data or information.

Processing circuitry 112 can execute instructions stored in memory 114. These instructions can cause processing circuitry 112 to process raw analyte data (or pre-processed analyte data) and arrive at a final calculated analyte level. Instructions stored in memory 114, when executed, can cause processing circuitry 112 to process raw analyte data to determine one or more of: a calculated analyte level, an average calculated analyte level within a predetermined time window, a calculated rate-of-change of an analyte level within a predetermined time window, and/or whether a calculated analyte metric exceeds a predetermined threshold condition. These instructions can also cause processing circuitry 112 to read and act on received transmissions, to adjust the timing of timing circuitry 115, to process data or information received from other devices (e.g., calibration information, encryption or authentication information received from display device 120, and others), to perform tasks to establish and maintain communication with display device 120, to interpret voice commands from a user, to cause communication circuitry 116 to transmit, and others. In embodiments where sensor control device 102 includes a user interface, then the instructions can cause processing circuitry 112 to control the user interface, read user input from the user interface, cause the display of information on the user interface, format data for display, and others. The functions described here that are coded in the instructions can instead be implemented by sensor control device 102 with the use of a hardware or firmware design that does not rely on the execution of stored software instructions to accomplish the functions.

Memory 114 can be shared by one or more of the various functional units present within sensor control device 102, or can be distributed amongst two or more of them (e.g., as separate memories present within different chips). Memory 114 can also be a separate chip of its own. Memory 114 is non-transitory, and can be volatile (e.g., RAM, etc.) and/or non-volatile memory (e.g., ROM, flash memory, F-RAM, etc.).

Communication circuitry 116 can be implemented as one or more components (e.g., transmitter, receiver, transceiver, passive circuit, encoder, decoder, and/or other communication circuitry) that perform the functions for communications over the respective communications paths or links. Communication circuitry 116 can include or be coupled to one or more antennae for wireless communication.

Power supply 111 can include one or more batteries, which can be rechargeable or single-use disposable batteries. Power management circuitry can also be included to regulate battery charging and monitor usage of power supply 111, boost power, perform DC conversions, and the like.

Additionally, an on-skin or sensor temperature reading or measurement can be collected by an optional temperature sensor. Those readings or measurements can be communicated (either individually or as an aggregated measurement over time) from sensor control device 102 to another device (e.g., display device 120). The temperature reading or measurement, however, can be used in conjunction with a software routine executed by sensor control device 102 or display device 120 to correct or compensate the analyte measurement output to the user, instead of or in addition to, actually outputting the temperature measurement to the user.

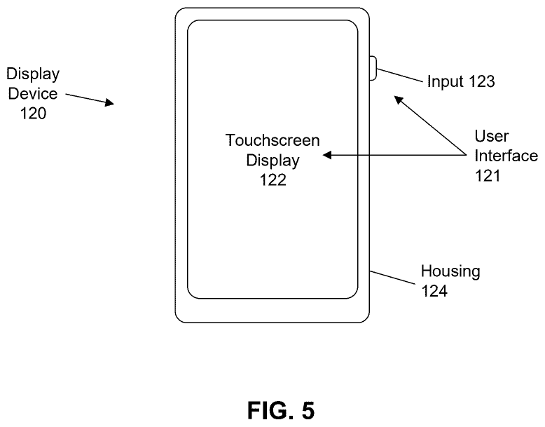

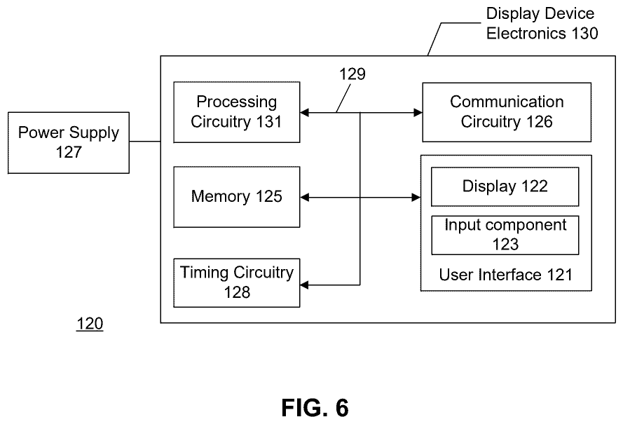

FIG. 5 is a schematic view depicting an example embodiment of display device 120. Here, display device 120 includes a user interface 121 and a housing 124 in which display device electronics 130 (FIG. 6) are held. User interface 121 can be implemented as a single component (e.g., a touchscreen capable of input and output) or multiple components (e.g., a display and one or more devices configured to receive user input). In this embodiment, user interface 121 includes a touchscreen display 122 (configured to display information and graphics and accept user input by touch) and an input component 123, both of which are coupled with housing 124.

Display device 120 can have software stored thereon (e.g., by the manufacturer or downloaded by the user in the form of one or more “apps” or other software packages) that interface with sensor control device 102, medication delivery device 152, and/or the user. In addition, or alternatively, the user interface can be affected by a web page displayed on a browser or other internet interfacing software executable on display device 120.

Display device 120 can be configured to display information pertaining to dose guidance system 100 to the user and accept or receive input from the user also pertaining to dose guidance system 100. Display device 120 can display recent measured analyte levels, in any number of forms, to the user. The display device can display historical analyte levels of the user as well as other metrics that describe the user's analyte information (e.g., time in range, ambulatory glucose profile (AGP), hypoglycemia risk levels, etc.). Display device 120 can display medication delivery information, such as historical dose information and the times and dates of administration, dose guidance system settings or parameters, or dose recommendations, among other information.

Display device 120 can also be configured to output alarms, alerts, or other notifications relating to dose guidance system, such as analyte data or medication delivery information, which may be visual, audible, tactile, or any combination thereof. Sensor control device 102 and/or medication delivery device 152 can also be configured to output alarms, or alert notifications in visible, audible, tactile forms or combination thereof. Further details and other display embodiments can be found in, e.g., U.S. Patent Publ. No. 2011/0193704, which is incorporated herein by reference in its entirety for all purposes.

Display device 120 can be dedicated for use with dose guidance system 100 (e.g., an electronic device designed and manufactured for the primary purpose of interfacing with an analyte sensor and/or a medication delivery device), as well as devices that are multifunctional, general purpose computing devices such as a handheld or portable mobile communication device (e.g., a smartphone or tablet), or a laptop, personal computer, or other computing device. Display device 120 can be configured as a mobile smart wearable electronics assembly, such as a smart glass or smart glasses, or a smart watch or wristband. Display devices, and variations thereof, can be referred to as “reader devices,” “readers,” “handheld electronics” (or handhelds), “portable data processing” devices or units, “information receivers,” “receiver” devices or units (or simply receivers), “relay” devices or units, or “remote” devices or units, to name a few.

FIG. 6 is a block diagram of an example embodiment of a display device 120 with display device electronics 130. Here, display device 120 includes user interface 121 including display 122 and an input component 123 (e.g., a button, actuator, touch sensitive switch, capacitive switch, pressure sensitive switch, jog wheel, microphone, speaker, or the like), processing circuitry 131, memory 125, communication circuitry 126 configured to communicate to and/or from one or more other devices external to display device 120), a power supply 127, and timing circuitry 128 (e.g., such as an oscillator and phase locked loop for providing a clock or other timing to components of sensor control device 102). Each of the components can be implemented as one or more different devices or can be combined into a multifunctional device (e.g., integration of processing circuitry 131, memory 125, and communication circuitry 126 on a single semiconductor chip). Display device 120 can be implemented in a highly interconnected fashion, where power supply 127 is coupled with each component shown in FIG. 6 and where those components that communicate or receive data, information, or commands (e.g., user interface 121, processing circuitry 131, memory 125, communication circuitry 126, and timing circuitry 128), can be communicatively coupled with every other such component over, for example, one or more communication connections or buses 129. FIG. 6 is an abbreviated representation of the typical hardware and functionality that resides within a display device and those of ordinary skill in the art will readily recognize that other hardware and functionality (e.g., codecs, drivers, glue logic) can also be included.

Processing circuitry 131 can include one or more processors, microprocessors, controllers, and/or microcontrollers, each of which can be a discrete chip or distributed amongst (and a portion of) a number of different chips. Processing circuitry 131 can include on-board memory. Processing circuitry 131 can interface with communication circuitry 126 and perform analog-to-digital conversions, encoding and decoding, digital signal processing and other functions that facilitate the conversion of data signals into a format (e.g., in-phase and quadrature) suitable for wireless or wired transmission. Processing circuitry 131 can also interface with communication circuitry 126 to perform the reverse functions necessary to receive a wireless transmission and convert it into digital data or information.

Processing circuitry 131 can execute software instructions stored in memory 125. These instructions can cause processing circuitry 131 to process raw analyte data (or pre-processed analyte data) and arrive at a corresponding analyte level suitable for display to the user. These instructions can cause processing circuitry 131 to read, process, and/or store a dose instruction from the user, and cause the dose instruction to be communicated to medication delivery device 152. These instructions can cause processing circuitry 131 to execute user interface software adapted to present an interactive group of graphical user interface screens to the user for the purposes of configuring system parameters (e.g., alarm thresholds, notification settings, display preferences, and the like), presenting current and historical analyte level information to the user, presenting current and historical medication delivery information to the user, collecting other non-analyte information from the user (e.g., information about meals consumed, activities performed, medication administered, and the like), and presenting notifications and alarms to the user. These instructions can also cause processing circuitry 131 to cause communication circuitry 126 to transmit, can cause processing circuitry 131 to read and act on received transmissions, to read input from user interface 121 (e.g., entry of a medication dose to be administered or confirmation of a recommended medication dose), to display data or information on user interface 121, to adjust the timing of timing circuitry 128, to process data or information received from other devices (e.g., analyte data, calibration information, encryption or authentication information received from sensor control device 102, medication dose information received from a smart injection pen or dose detection device, such as a smart pen cap, and others), to perform tasks to establish and maintain communication with sensor control device 102, to interpret voice commands from a user, and others. The functions described here that are coded in the instructions can instead be implemented by display device 120 with the use of a hardware or firmware design that does not rely on the execution of stored software instructions to accomplish the functions.

Memory 125 can be shared by one or more of the various functional units present within display device 120, or can be distributed amongst two or more of them (e.g., as separate memories present within different chips). Memory 125 can also be a separate chip of its own. Memory 125 is non-transitory, and can be volatile (e.g., RAM, etc.) and/or non-volatile memory (e.g., ROM, flash memory, F-RAM, etc.).

Communication circuitry 126 can be implemented as one or more components (e.g., transmitter, receiver, transceiver, passive circuit, encoder, decoder, and/or other communication circuitry) that perform the functions for communications over the respective communications paths or links. Communication circuitry 126 can include or be coupled to one or more antennae for wireless communication.

Power supply 127 can include one or more batteries, which can be rechargeable or single-use disposable batteries. Power management circuitry can also be included to regulate battery charging and monitor usage of power supply 127, boost power, perform DC conversions, and the like.

Display device 120 can also include one or more data communication ports (not shown) for wired data communication with external devices such as computer system 170, sensor control device 102, medication delivery device 152, or dose detection device 160. Display device 120 may also include an integrated or attachable in vitro glucose meter, including an in vitro test strip port (not shown) to receive an in vitro glucose test strip for performing in vitro blood glucose measurements.

Basal insulin titration algorithms and methods as described herein may be executed by one or more components of dose guidance system 100, such as by one or more of a glucose monitoring device, a medication delivery device (e.g., patch pump, infusion pump, smart injection pen), an accessory for a medication delivery device (e.g., dose detection device, smart pen cap), a display device (such as a smartphone or dedicated handheld receiver), or a remote computer, server, or cloud, and combinations thereof.

Dose guidance systems or algorithms for titrating a basal insulin dose as described herein may include setting an initial basal insulin dose. The initial basal insulin dose may be manually entered by the user (such as in conjunction with or as directed by an HCP). The user may select from a list or manually type in a dose amount using an input of a display device of the dose guidance system. If the user is already on basal insulin therapy when starting to use the dose guidance system, the initial dose may be set to the user's current (i.e., most recent) basal insulin dose. In some examples, the user's HCP may enter the initial basal insulin dose.

Alternatively, the initial basal insulin dose may be set by the dose guidance system to a default or preprogrammed value. The initial basal insulin dose may be a predetermined value for all users. The initial basal insulin dose may be a default value based on population data. The initial basal insulin dose may be a conservative value to avoid inducing hypoglycemia in patients initiating basal insulin therapy. The initial basal insulin dose may be at least around 8 units and/or no greater than around 12 units, for example, 8 units to 12 units, and may be around 10 units.

Alternatively, dose guidance system 100 may determine the initial basal insulin dose based on patient data, such as based on the patient's body weight. The user may enter a body weight into the dose guidance system, such as by an input of a display device, by receipt of the body weight from a connected scale, or by retrieving the body weight from an electronic medical record (EMR). The dose guidance system may store a dose to body weight ratio in memory. The dose to body weight ratio may be at least around 0.1 units/kg/day and/or no greater than around 0.3 units/kg/day, for example in a range of 0.1 units/kg/day to 0.3 units/kg/day. The dose to body weight ratio may be selected consistent with ADA guidelines. The basal insulin dose may be determined by Equation 1:

$$Dose = k \times weight$$

where k is the dose to body weight ratio and may be in a range of 0.1 units/kg/day to 0.3 units/kg/day, and may be 0.2 units/kg/day, weight is the user's body weight in kilograms, and the Dose is the initial basal insulin dose in units.

For example, if a user's body weight is 72 kg, and the dose to body weight ratio is 0.2 units/kg/day, the initial basal insulin dose is 14.4 units. The dose may be rounded to the nearest whole unit dose by dose guidance system 100. In the above example, the dose may be rounded to 14 units. The dose may be rounded down to the nearest whole unit for safety to avoid increasing the dose beyond what is recommended based on the user's body weight (e.g., 14.7 units is rounded down to 14 units rather than up to 15 units). The dose to body weight ratio may be preprogrammed into the dose guidance system. The dose to body weight ratio may be settable by the HCP, such as to adjust the aggressiveness of treatment, with larger ratio being a more aggressive treatment. The dose to body weight ratio may be adjustable within a range of values, such as within a range of 0.1 units/kg/day to 0.3 units/kg/day.

The user may update his or her body weight in the dose guidance system at any time. The user's weight may also be updated in his or her EMR and communicated to dose guidance system 100. When the user's body weight is updated (e.g., by the user or via the EMR), the basal insulin dose may be reset based on the newly entered body weight. The dose guidance system 100 may reset titration and begin titrating based on the reset basal insulin dose. Dose guidance system 100 may determine and track a ratio of the current basal insulin dose to the user's body weight. The ratio of the basal insulin dose to body weight may be used to determine overbasalization, as discussed in further detail herein.

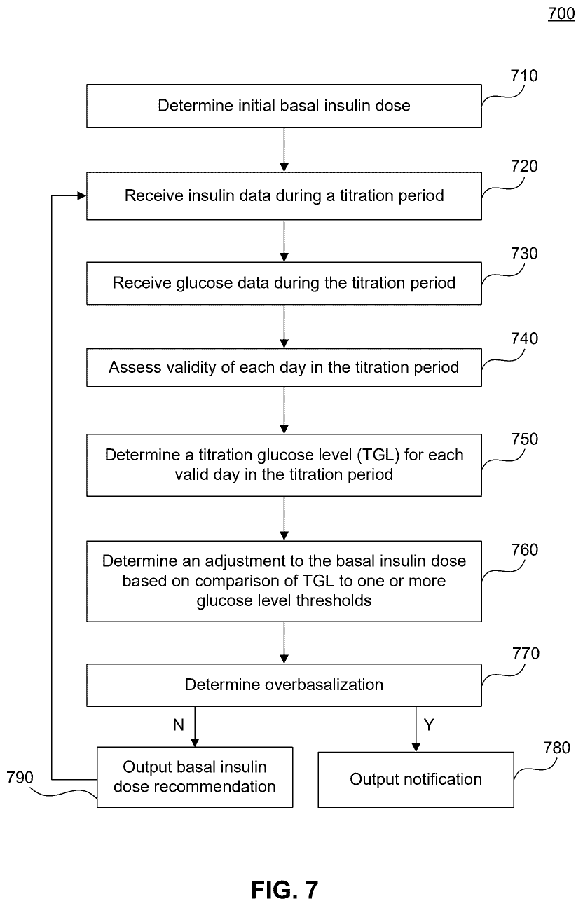

An exemplary method for initiating and titrating a basal insulin dose is shown in FIG. 7. An initial basal insulin dose is set or determined 710. The dose guidance system receives insulin data during the titration period 720. The insulin data may be received by the user manually entering the insulin dose administered into the dose guidance system, such as by manually entering dose information into a mobile application on a display device. In some examples, the insulin data for the titration period is received at or after an end of the titration period. The insulin dose may be automatically received from a dose detection device or a medication delivery device.

The dose guidance system may receive glucose data during (or for) the titration period 730. The dose guidance system may receive the glucose data at or after an end of the titration period. The glucose data may be collected by a glucose monitoring device, such as a CGM worn by the patient. The dose guidance system may receive glucose data directly from the glucose monitoring device, or may retrieve glucose data from a memory, or from a cloud, remote server, or the like.

The dose guidance system may optionally determine a validity of each day of glucose data in the titration period 740. In some examples, the validity of each day is automatically determined upon the completion of each day. In other examples, the validity of each day is determined upon the completion of a predetermined period of time (e.g., two, three, seven, fourteen, or fifteen days). A day may be defined as the time between two basal doses (e.g., in a scenario where one basal dose is administered for each calendar day). A titration glucose level (TGL) is determined for each valid day in the titration period 750. In some embodiments, step 740 is omitted (e.g., the validity of each day is not determined by the dose guidance system) and a TGL value is determined for each day in the titration period.

Upon the termination of the titration period, the dose guidance system may determine an adjustment to the basal insulin dose based on comparison of the TGL for each day (or each valid day) in the titration period to one or more glucose level thresholds 760. The dose adjustment may include a determination to increase the dose, decrease the dose, or maintain the dose. The dose adjustment may include the specific amount of the adjustment, as a whole number or as a percentage of the current basal insulin dose.

The dose guidance system may optionally detect overbasalization 770. Overbasalization may be based on a ratio of the basal insulin dose to the user's body weight. If overbasalization is detected, the dose guidance system may output a notification to the user 780. The notification may include an indication of overbasalization, a recommendation to consult with the user's HCP to discuss escalating therapy (e.g., to add rapid-acting insulin, among other therapies), or both. The system may stop providing recommendations to increase the basal insulin dose, but may still titrate to decrease the basal insulin dose. The system may alternatively allow for continued titration to increase the basal insulin dose up to a predetermined limit. If overbasalization is not detected, the dose guidance system may recommend the basal insulin dose based on the determined adjustment 790. The dose guidance system repeats steps 720 to 790 to titrate the basal insulin dose over time. In some embodiments, determining overbasalization 770 is omitted, and the determined adjustment to the basal insulin dose is output to the user at operation 790.

The dose guidance systems and titration algorithms described herein may receive glucose data and insulin data over a titration period. A titration period may correspond to a number of days of data to be collected in order to determine a titration recommendation, such as to increase, maintain or decrease the basal insulin dose. The titration period may be predetermined by the dose guidance system. The titration period may be one or more days. For example, the titration period may be around 2 or more days, around 3 or more days, less than or equal to around 7 days, less than or equal to around 5 days, and/or less than or equal to around 3 days. For example, the titration period may be 2 to 7 days, 2 to 5 days, or 2 to 3 days. The more days of glucose data are collected, the more accurate the glucose data may be of the user's glucose control. However, the longer the titration period, the less frequently the basal insulin dose is titrated, which can result in slowed improvement to glucose control. If the titration algorithm includes a validity assessment, the titration period may correspond to a number of valid days of data to be collected in order to determine a titration recommendation. For example, if the titration period is three days of valid data, and three days of data have been collected with one day of data being deemed invalid, additional day or days or data must be collected until a third day of valid data is obtained. The titration period may be a period consistent with ADA guidelines. In some examples, the titration period may be set and/or updated by the HCP. In other examples, the titration period is preset (e.g., preprogrammed or factory set) and cannot be changed by the user or HCP.

The dose guidance system may receive insulin data by manual user entry, such as via an input of a display device, or from a medication delivery device (e.g., pump or smart pen) or dose detection device (e.g., smart pen cap). The dose guidance system may receive glucose data collected by a glucose monitoring device. The glucose data may be received directly from the glucose monitoring device, or may be received from a cloud or server on which the glucose data is stored.

The dose guidance system may divide or segment the titration period into one or more days. The days may be based on administration of basal insulin doses. A day may be determined based on a time period between basal insulin doses. A first day may begin at the time of administration of a first basal insulin dose and may end upon administration of a second or subsequent basal insulin dose. For example, if a first dose is administered at 8:00 PM on Tuesday, and a consecutive, second dose is administered at 9:00 PM on Wednesday, the day is 25 hours. The glucose data may be divided or segmented into each day. The glucose data for each day may be analyzed to determine a titration recommendation as further discussed below.

Each day in the titration period may be assessed for validity. Only days that are determined to be satisfy the validity assessment are used in the titration determination. Users may not take doses at precisely the same time each day, and may miss a dose for one or more days. As a result, the time between doses and the amount of glucose data between doses can vary. If the time between doses is too short, insufficient glucose data may be collected on which to make a titration determination, and conversely if the time between doses is too long, the titration determination may rely on glucose data that is not impacted by the dose. Thus, there is a need to ensure sufficient glucose data is available to measure the user's glucose response and to rely on glucose data that is reflective of the user's glucose response to the administered dose.

In some embodiments, the validity assessment may be based on a duration of the day (e.g., the duration between basal insulin doses). The validity assessment may require the day to be greater than a minimum duration, less than a maximum duration, or both. The validity assessment may be performed at the end of a day (e.g., upon administration of a second basal insulin dose). In some embodiments, the validity assessment may be performed after a predetermined time period following administration of a dose.

In some embodiments, the validity assessment includes a determination of whether the duration of each day is greater than a minimum duration. In some embodiments, the minimum duration may be preset or predetermined. The minimum duration may be at least around 14 hours and/or no greater than around 20 hours, for example the minimum duration may be in a range of 14 hours to 20 hours, and may be for example around 18 hours. In some examples, the minimum duration may be dynamic or personalized to the user. For example, the minimum duration can be a function of the user's total daily dose (TDD), insulin dose regimen, and/or body weight. In some embodiments, if the duration of the day is less than the minimum duration, the day is assessed to be invalid and is not used in the titration determination. In an example, if a day is 16 hours and the minimum duration is 18 hours, the day of data will be determined to be invalid and will not be used for a titration determination.

In some embodiments, if the day is less than the minimum duration, the day may still be considered valid for determinations to decrease the basal insulin dose, but may not be used for a determination to increase the basal insulin dose. This is because there is greater risk when increasing a dose amount, as the higher dose may result in lower glucose levels and hypoglycemia, whereas there is relatively less risk to decrease a dose amount. The dose guidance system may optionally include a second minimum duration that is lower than the first minimum duration. The second minimum duration, for example, may be at least around 12 hours and/or no greater than around 16 hours. For example, the second minimum duration may be in a range of 12 hours to 16 hours, and may be around 13 hours. In some examples, the second minimum duration may be lower than 12 hours (e.g., 4, 6, 8 hours)—for example, when the user's insulin regimen includes two or more insulin injections in a 24-hour period. If the duration of the day is below the first minimum duration but above the second minimum duration, the day of data may be used for a determination to decrease the basal insulin dose but not for a determination to increase the basal insulin dose. If the duration of the day is below the second minimum duration, the day is not used for any titration determinations.

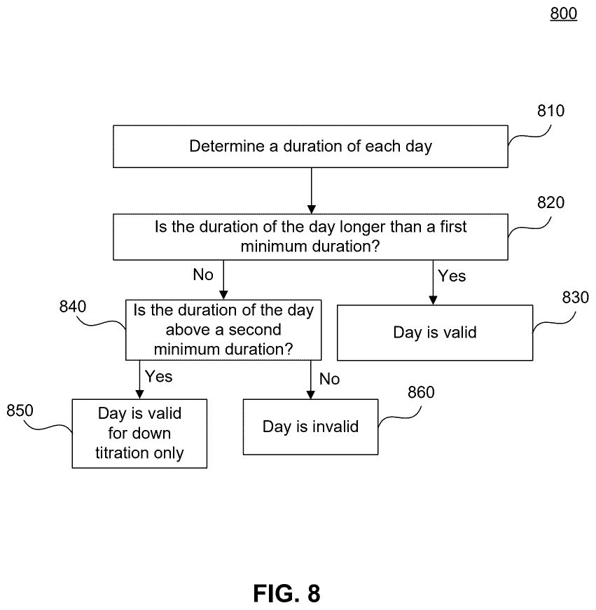

An exemplary validity assessment is shown in FIG. 8. A duration of each day of data is determined 810. The duration of the day is based on the time period between basal insulin doses. The dose guidance system determines if the duration of the day is longer than a first minimum duration 820. If so, the day is valid 830 and the day of data can be used in the titration determination, subject to other validity assessments (e.g., an assessment for a maximum duration). If the duration is below the first minimum duration, the dose guidance system determines if the duration is above a second minimum duration 840. The second minimum duration is shorter than the first minimum duration. If the duration is greater than the second minimum duration, the day of data is valid for down titration (e.g., for determining to decrease the dose) 850. If the duration is below the second minimum duration, the day is invalid for all titration determinations 860.

The validity assessment may include comparing the duration of the day to a maximum duration. The maximum duration may be at least around 28 hours and/or no greater than around 36 hours, for example the maximum duration may be in a range of 28 hours to 36 hours, or in a range of 30 hours to 34 hours, and may be around 30 hours. In some embodiments, if the duration exceeds the maximum duration, the day is invalid and is not used in a titration determination. In some embodiments, if the duration of the day exceeds the maximum duration, the day of data is valid but is limited in duration up to the maximum duration. For example, if the day is 36 hours, and the maximum duration is 30 hours, the day is valid but the day is limited to 30 hours. The day may be limited to the first 30 hours following administration of the dose, with the 6 hours after the first 30 hours not considered for purposes of the titration determination.

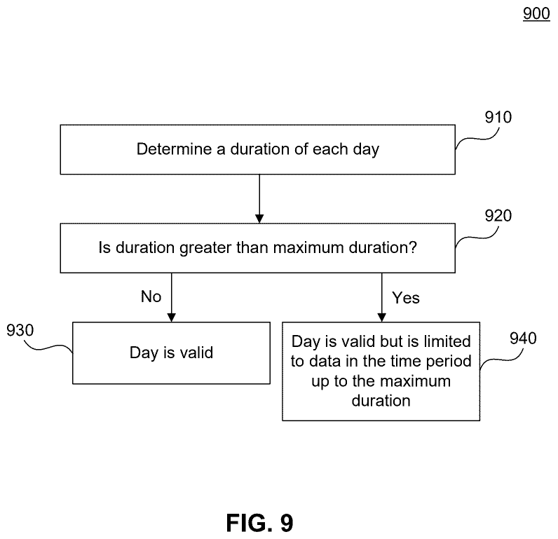

An exemplary validity assessment is shown in FIG. 9. A duration of each day is determined 910. The duration of each day may be determined based on the times of the basal insulin doses during the titration period. The dose guidance system determines if the duration for each day is greater than a first maximum duration 920. If not, the day of data is valid 930, subject to other validity assessments (e.g., an assessment for a minimum duration). If the duration is greater than the maximum duration, the day of data is valid 940, but is limited to the amount of time up to the maximum duration.

The validity assessment may include determining if the day has a duration in a predetermined range between a minimum and a maximum duration, wherein the day is valid if the duration is within the predetermined range. The range may be, for example, around 12 hours to around 36 hours, around 16 hours to around 30 hours, or around 18 hours to around 28 hours, among other ranges.

Each day of data may be assessed for validity based on the glucose data collected. Glucose levels from the glucose monitoring device are collected throughout the course of a day (e.g., every 1 minute, every 3 minutes, every 5 minutes, among other intervals). The dose guidance system may assess the gaps or missing glucose data in the day and determine a metric based on the amount of glucose data collected. Gaps in glucose data may occur where the display device is out of communication range of the glucose monitoring device, when the glucose monitoring device has expired and the user does not immediately apply a new glucose monitoring device, due to the glucose monitoring device falling off or becoming dislodged, due to damage or malfunction of the glucose monitoring device or display device, among other factors. Titrating an insulin dose based on incomplete glucose data can reduce the accuracy of the titration determination. However, as a practical matter it can be common to have gaps in data, and refusing to titrate unless all glucose data is present could be very limiting and may greatly slow the titration process. Thus, there is a need to ensure sufficient glucose data is available to accurately make a titration determination, while providing flexibility to make a titration determination based on available data. In some embodiments, a day of data is assessed to be valid when the amount or percent of glucose data in the day meets or exceeds a glucose data threshold. The glucose data threshold may be, for example, 70%, 75%, or 80%, among other values. This validity assessment based on the amount of glucose data or completeness of glucose data ensures that sufficient data is available from which to determine the user's glucose response.

In some embodiments, the glucose data threshold may depend on the titration glucose level (TGL), as discussed in further detail below. If the TGL is below a lower glucose level threshold (such as below 80 mg/dl, 70 mg/dl, or 54 mg/dl among other values), the day of data is valid regardless of the amount of glucose data collected. This is to ensure that low glucose data is accounted for in the titration determination. If the TGL is above the lower glucose level threshold (such as above 80 mg/dl), then the day of data is assessed to be valid if the amount of glucose data collected meets or exceeds the glucose data threshold.

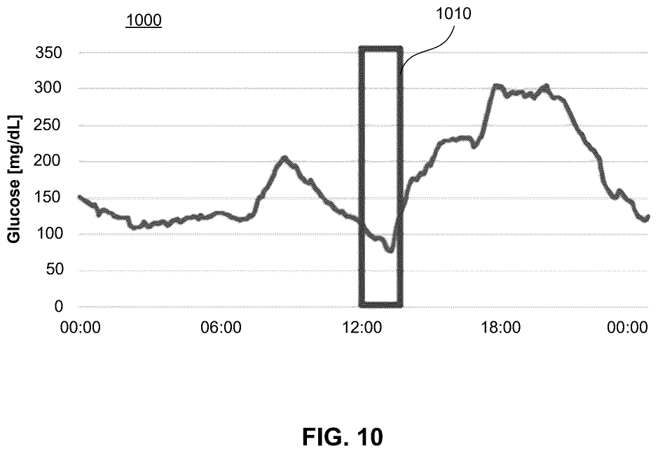

The titration determination may be based on analysis of the glucose data for each day in the titration period (or for each valid day in the titration period where a validity assessment is implemented). The dose guidance system may analyze the glucose data to determine a TGL for each day in the titration period. The TGL may correspond to a fasting glucose level. The TGL may be based on a lowest glucose level in the day. Fasting glucose level can be determined based on a glucose level in an overnight period when there is the largest gap between meals. However, an overnight glucose level may not be an accurate indicator of fasting glucose level for all users, such as users who perform shift work, while users travel to different time zones, or who otherwise keep irregular schedules. Thus, the TGL determined as described herein may provide an improved measure of a fasting glucose level for a user irrespective of their schedule. The TGL may be determined by dividing the glucose levels in one day 1000 into a plurality of time blocks 1010, as shown for example in FIG. 10. The time blocks may be non-overlapping. For example, a 24-hour day may be divided in 24 consecutive 1-hour blocks. Each time block 1010 may be fixed amount of time, such as at least around 0.5 hours and/or no greater than around 2 hours, for example 0.5 hours to 2 hours, and may be around 1 hour. It is understood that the time blocks may be longer or shorter amounts of time. Shorter time blocks may be more subject to noise and outlier data, whereas longer time blocks may not be reflective of a low glucose event. The average glucose level for each time block is calculated. The TGL is determined to be the lowest average glucose level of the plurality of time blocks. Alternatively, other measures of central tendency may be computed for each time block and used to assess the TGL. In another example, a median glucose level may be computed for each time block, and the TGL corresponds to the lowest median value of the time blocks in the day.

The TGL for each day in the titration period is compared to one or more glucose level thresholds to determine an adjustment of the basal insulin dose. The TGL is compared to one or more lower glucose level thresholds. If the TGL is below the lower glucose level threshold, the titration determination may be to decrease the dose. The titration determination may first determine whether to decrease the dose before determining whether to increase the dose. In some embodiments, the dose may only be increased if there are no days in the titration period in which the TGL is below the lower glucose level threshold. This provides a safety measure to avoid increasing the dose where there is a risk of hypoglycemia. The TGL may be compared to one or more upper glucose level thresholds. If the TGL is above the upper glucose level threshold, the titration determination may be to increase the basal insulin dose.

In an example, the lower glucose level threshold may be at least 70 mg/dl, for example, 80 mg/dl, and the upper glucose level threshold may be no greater than around 130 mg/dl, for example 109 mg/dl or 99 mg/dl. The lower glucose level threshold and upper glucose level threshold may be based on a target range for glucose levels. The target glucose range may be greater than or equal to around 70 mg/dl or greater than or equal to around 80 mg/dl. The target glucose range may be less than or equal to around 130 mg/dl, less than or equal to around 109 mg/dl, or less than or equal to around 99 mg/dl. The target glucose range may be from 70 mg/dl to 130 mg/dl, 80 mg/dl to 130 mg/dl, 70 mg/dl to 109 mg/dl, or 80 mg/dl to 99 mg/dl, among other ranges. However, it is understood that the glucose level thresholds may be other values. In some embodiments, the lower glucose level and upper glucose level thresholds, may be set based on patient characteristics, such as the patient's gender, body weight, age, hypoglycemia risk, fear of hypoglycemia, and comorbidities, among other factors.

In some embodiments, the glucose level thresholds may be fixed by the dose guidance system. Alternatively, the dose guidance system may allow a user (such as the HCP) to set the glucose level thresholds. The glucose level thresholds may be adjusted depending on the aggressiveness of treatment. The titration determination may require multiple days in which the TGL is below the lower glucose level threshold or above the upper glucose level threshold to determine to decrease or increase the basal insulin dose, respectively. This helps to ensure sufficient amount of data is relied on to determine the user's glucose control and the user's response to a given basal insulin dose.

The titration period (the number of days) to recommend a decrease to the basal insulin dose may be the same or may differ from the titration period (number of days) to recommend an increase to the basal insulin dose. In some embodiments, the titration period (number of days) to increase the basal insulin dose may be greater than the titration period (number of days) to decrease the basal insulin dose. For example, the titration period to decrease the basal insulin dose may be two days, whereas the titration period to increase the basal insulin dose may be three days. This helps to quickly decrease the basal insulin dose to alleviate low glucose level conditions when the basal insulin dose is too high. This also helps to more slowly increase the basal insulin dose for safety to avoid inducing hypoglycemia, and to have greater confidence in the glucose data prior to increasing the basal insulin dose.

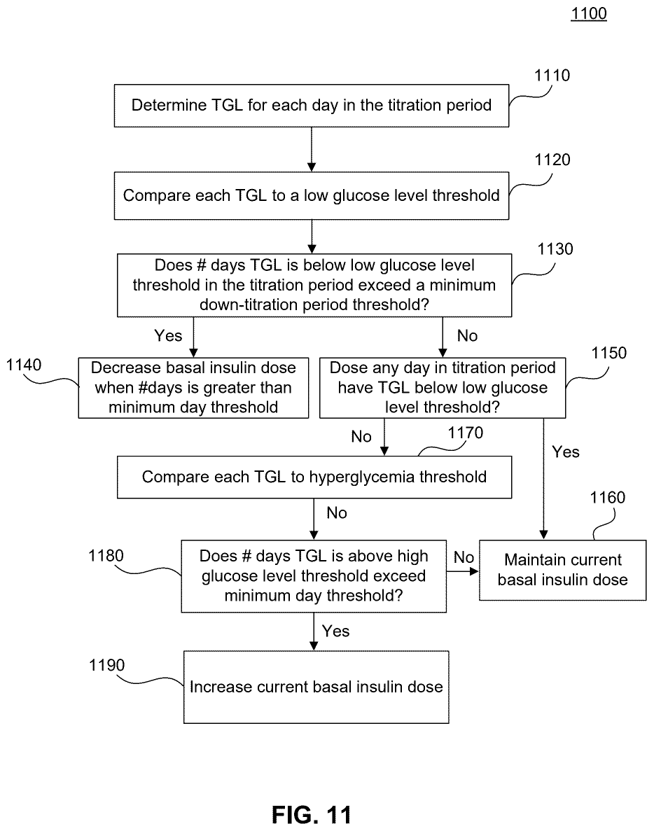

An exemplary method for a titration determination 1100 is shown in FIG. 11. A TGL is determined for each day (or each valid day) in the titration period 1110. The TGL for each day is compared to a lower glucose level threshold 1120. The number of days in the titration period in which the TGL is below the lower glucose level threshold is determined 1130. If the number of days the TGL is below the lower glucose level threshold meets or exceeds a minimum down-titration period threshold (e.g., 2 days, 3 days, 4 days, among others), the titration determination is to decrease the basal insulin dose 1140. If the number of days TGL is below the lower glucose level threshold does not meet or exceed the minimum down-titration period threshold, the dose guidance system may determine whether at least one day in the titration period has a TGL below the lower glucose level threshold 1150. If so, the titration determination is to maintain the current basal insulin dose 1160. If no days have a TGL below a lower glucose level threshold, a determination is made whether to increase the basal insulin dose. The dose guidance system compares the TGL for each day to an upper glucose level threshold 1170. The dose guidance system determines whether the number of days the TGL is above the upper glucose level threshold meets or exceeds a minimum up-titration period threshold (e.g., 2 days, 3 days, 4 days, among others) 1180. If so, the determination is to increase the basal insulin dose 1190. Otherwise, the determination is to maintain the current basal insulin dose 1160. The titration method 1100 may be performed at medication delivery device 152, display device 120, cloud network 190, computer system 170, or trusted computer system 180 (see, e.g., FIG. 1), or a combination thereof.

In an example, if the minimum down-titration period threshold is 2 days, a first day in the titration period has a TGL below the lower glucose level threshold, and a second day of the titration period has a TGL below the lower glucose level threshold, the titration recommendation can be made based on the two days of data to decrease the basal insulin dose.

In another example if a first day has a TGL below the lower glucose level threshold, and a second day has a TGL above the lower glucose level threshold, a titration determination cannot yet be made, and a third day of data is needed. If the third day includes a TGL below the lower glucose level threshold, the minimum down-titration period threshold is satisfied and the titration recommendation is to decrease the basal insulin dose. Alternatively, if the titration period is three days, and the third day has a TGL above the lower glucose level threshold, the titration recommendation is to maintain the current basal insulin dose.

In some embodiments, additional glucose level thresholds may be present. For example, the dose guidance system may include first and second upper glucose level thresholds, wherein the second upper glucose level threshold is higher than the first upper glucose level threshold. For example, the first upper glucose level threshold may be 130 mg/dl and the second upper glucose level threshold may be 180 mg/dl. If the TGL for a minimum up-titration period threshold (e.g., 2 days, 3 days) is greater than the first upper glucose level threshold, but is below the second upper glucose level threshold, the basal insulin dose is increased by a first amount. If the TGL for a minimum up-titration period threshold is greater than the second upper glucose level threshold, the basal insulin dose is increased by a second, greater amount. It is understood that additional upper or lower glucose level thresholds may be present to further scale the amount of increase or decrease of the basal insulin dose depending on comparison of the TGL to glucose level thresholds, with lower TGLs corresponding to larger decreases in the dose, and higher TGLs corresponding to larger increases in the dose. This helps to increase the speed of the titration by allowing for larger dose adjustments where the user's glucose levels are very low or very high relative to the glucose level thresholds.

The dose guidance system may further determine the amount of the adjustment in additional to the determination to decrease, maintain or increase the basal insulin dose. If a number of days in the titration period in which TGL is below the lower glucose level threshold meets the minimum down-titration period threshold (e.g., 2 or more days), the titration recommendation is to decrease the basal insulin dose amount by a first amount. The first amount may be a percentage of the current basal insulin dose, and may be at least around 5%, no greater than around 20%, in a range of 5% to 20%, and may be around 10%, or may be an absolute amount, such as at least around 1 unit, no greater than around 4 units, in a range of 1 unit to 4 units, and may be around 2 units. The amount may be consistent with ADA guidelines. If the number of days win which TGL is below a second lower glucose level threshold exceeds a minimum down-titration period threshold (e.g., 2 days, 3 days), the titration recommendation is to decrease the basal insulin dose by a second amount. The second amount be greater than the first amount. The second amount may be a percentage of the current basal insulin dose, or may be an absolute amount. The second amount may be a multiple of the first amount. For example, if the first amount is 5%, the second amount may be 10%. It is understood that dose guidance system may include additional or fewer lower glucose level thresholds, e.g., a third lower glucose level, a fourth lower glucose level, each having a corresponding dose amount adjustment.

If there are no days with a TGL less than the lower glucose level threshold, and there the number of days in which TGL meets or exceeds the upper glucose level threshold meets or exceeds a minimum up-titration period threshold, the titration recommendation may be to increase the basal insulin dose by a third amount. The third amount may be a percentage of the current basal insulin dose, and may be at least around 5%, no greater than around 20%, in a range of 5% to 20%, and may be 10%, or may be an absolute amount, such as at least around 1 unit, no greater than around 4 units, in a range of 1 unit to 4 units, and may be around 2 units. The third amount may be consistent with ADA guidelines.

If there are no days with a TGL less than the lower glucose level threshold and a number of days in the titration period having a TGL that meets or exceeds a second upper glucose level threshold meets or exceeds a minimum up-titration period threshold, wherein the second upper glucose level threshold is greater than the upper glucose level threshold, the titration determination may be to add a fourth amount to the basal insulin dose. The fourth amount may be greater than the third amount. For example, if the third amount is 10%, the fourth amount may be 20%. In another example, if the third amount is 2 units, the fourth amount may be 4 units. The fourth amount may be a multiple of the third amount, such as greater than 1 times, at least around 1.25 times, 1.25 times, 1.5 times, or 2 times the third amount. It is understood that dose guidance may include additional or fewer upper glucose level thresholds and corresponding dose amount adjustments.

For all other cases, the titration determination may be to maintain the current basal insulin dose.

The amount of the adjustment to the basal insulin dose may be determined based on a look-up table. An exemplary basal insulin titration look-up table is shown below in Table 1.

TABLE 1
Basal Insulin Dose Adjustment Look-up Table

| Titration Determination based on TGL | Basal Dose Adjustment |
|---|---|
| 2 or more days with TGL less than lower | Subtract the higher of 2 U |
| glucose level threshold | or 10% from current dose |
| 0 days with TGL less than lower glucose | Add the higher of 2 U |
| level threshold AND 2 or more days with | or 10% to current dose |
| TGL greater than first upper glucose | |
| level threshold | |
| 0 days of TGL less than the lower glucose | Add the higher of 4 U |
| level threshold AND 2 or more days with | or 20% to current dose |
| TGL greater than second upper glucose | |
| level threshold (and greater than first | |
| upper glucose level threshold) | |
| All other conditions (including 1 day with | Maintain current dose |
| TGL below lower glucose level threshold) | |

The titration recommendation may be output to the user. The titration recommendation may display the amount of the adjustment to the most recent dose, the new, titrated dose amount, or both. The titration recommendation may be displayed on a display device of the user, such as on a mobile application on a user's smartphone or handheld device. The titration recommendation may be displayed on a medication delivery device or accessory (e.g., on a smart pen cap). The basal insulin dose may be automatically administered by the medication delivery device or may require manual administration by the user. The titration recommendation may be output as an alert or notification on the display device. The titration recommendation may be output upon the determination of a new dose amount. The new dose amount may be output immediately upon determination of a new dose amount. The titration recommendation or recommended adjustment to the basal insulin dose may be recommended automatically at an end of the titration period and without user input.

In some embodiments, the titration recommendation may be output after a delay period. In some examples, the new dose amount may be determined and/or output after the delay period. The delay period may be a period of time following a time of administration of a previous (most recent) basal insulin dose, such as 4 hours to 20 hours, 6 hours to 18 hours, or 8 hours to 12 hours after the previous dose. This helps to prevent alerting the user to a new dose shortly after the user has already administered a dose, which may confuse the user and may cause the user to believe he or she should administer another dose. The delay period is also configured to ensure that the user receives the new dose alert before the time for the next basal insulin dose, so that the user administers the next basal insulin dose at the new dose amount. Alternatively, the new dose amount is not output until the user requests dose guidance from the dose guidance system. The dose guidance system may display the recommended basal insulin dose at that time.

The previous dose amount may be displayed along with the new dose amount. This may help to alert the user to the fact that the dose amount has changed. The amount of the adjustment may also be displayed to the user so that the user may understand how their dose has been adjusted (e.g., +1 unit, −1 unit). In some embodiments, only the new dose amount is displayed. This may help to avoid any confusion in the dose to be administered.

In some embodiments, the dose guidance system may be configured to detect overbasalization. Overbasalization may be a condition in which too much basal insulin is administered to the user without improvement to glucose control. When basal insulin is optimally titrated and glucose control is not achieved, the patient may progress to additional therapies, such as to include prandial insulin or rapid-acting insulin doses. Thus, detecting overbasalization can help to determine when further titration of the basal insulin dose is no longer effective in improving glucose control, and the patient should progress to additional therapies.

The dose guidance system may titrate the basal insulin dose based on the user's glucose data and insulin data as described herein. As the dose guidance system titrates the dose, the system may detect overbasalization. Overbasalization may be based on user data, glucose data, insulin data, or a combination thereof. In some embodiments, the user's body weight may be used to determine overbasalization.

Overbasalization may be determined based on a ratio of the current basal insulin dose to body weight. Overbasalization may be determined when the dose to body weight ratio exceeds a dose to body weight ratio threshold. In some embodiments, the threshold may be at least around 0.4 units/kg/day and/or no greater than around 0.6 units/kg/day, for example, in a range of 0.4 units/kg/day to 0.6 units/kg/day, and may be around 0.5 units/kg/day.

The dose guidance system may prompt the user to enter his or her body weight, such as during set-up of the dose guidance system. The dose guidance system may periodically prompt the user to input a body weight, such as once a week, once every month, once every two months, among other intervals, to ensure the body weight is accurate. The user's weight may also be updated in her or her Electronic Medical Record (EMR) and communicated to dose guidance system. As the dose guidance system titrates the basal insulin dose, the dose guidance system may determine if the adjustment to the dose would result in a ratio of basal insulin to body weight of 0.5 units/kg/day or more.

The dose guidance system may detect overbasalization based on alternate or additional measures. Overbasalization may be detected based on the TGL being below a lower glucose level threshold. The system may alternatively or additionally detect overbasalization when a change in the user's overnight glucose levels-sometimes referred to as bedtime to early morning (“BEAM”)—is greater than an overnight glucose level change threshold, e.g., 50 mg/dl, 60 mg/dl, or 70 mg/dl. Overbasalization may be determined based on a difference between a peak glucose level in a day and the TGL exceeding a glucose difference threshold. Alternatively or additionally, overbasalization may be determined based on a glucose management indicator (GMI) or a coefficient of variation (CV), such as the GMI or CV exceeding a predetermined threshold value, respectively. When the basal insulin dose to body weight ratio exceeds the threshold for overbasalization, the system may generate a report that includes the user's glucose metrics and insulin data for review by an HCP. The report may include one or more of the BEAM value, GMI, and CV, among other metrics, so that the HCP may review and determine whether to continue basal insulin therapy alone or progress to additional therapies.

If overbasalization is detected, the dose guidance system may not recommend a basal insulin dose that exceeds the ratio of basal insulin to body weight threshold. Once the ratio of basal insulin to body weight threshold is reached, the dose guidance system may no longer recommend increases to the basal insulin dose. The dose guidance system may, however, still determine decreases to the basal insulin dose. If the ratio of basal insulin to body weight threshold has been met, and the dose recommendation is to decrease the dose amount, the dose guidance system may subsequently titrate to recommend a dose increase up to the ratio of basal insulin to body weight threshold. In some embodiments, once the ratio of basal insulin to body weight threshold is reached, the dose guidance system may continue to recommend increases to the basal insulin dose up to a second ratio of basal insulin to body weight threshold. For example, the basal insulin to body weight threshold may be 0.5 U/kg/day and the second basal insulin to body weight threshold may be 0.8 U/kg/day. Alternatively, once the basal insulin to body weight threshold is reached, the dose guidance system may continue to recommend increases to the basal insulin dose based on one or more glucose metric thresholds. The glucose metric may include a measure of glucose variation, such as interquartile range, standard deviation, or CV, among others. In an example, the dose guidance system may continue to titrate the basal insulin dose to increase the dose amount when the glucose variation is less than a threshold amount, such as a glucose variation in a range of 30% to 40%, for example around 36%, among other values.

When overbasalization is determined by the dose guidance system using any of the above methods, the system may output a notification to the patient. The notification may indicate that the maximum basal insulin dose is reached or that overbasalization is detected. The notification may recommend that the patient discuss treatment with their HCP. In some examples, the dose guidance system may notify the user's HCP. The notification may recommend escalating therapy to include one or more additional medications, such as rapid-acting insulin doses for one or more meals, or glucagon-like peptide 1 (GLP-1) agonists, among other medications. The system may alternatively or additionally output a notification to a display device of the HCP or to an application or interface accessible by the HCP such that the HCP can review the patient's therapy.

In some embodiments, the dose guidance system may consider additional factors in titrating a basal insulin dose. Dose guidance system may titrate the basal insulin dose based on detection of low glucose events. The dose guidance system may recommend decreasing a basal insulin dose when a low glucose event is detected. When the patient experiences a low glucose event, it may be desirable to decrease the basal insulin dose to prevent further low glucose events and associated symptoms. This may improve patient safety and may help to quickly address overbasalization.

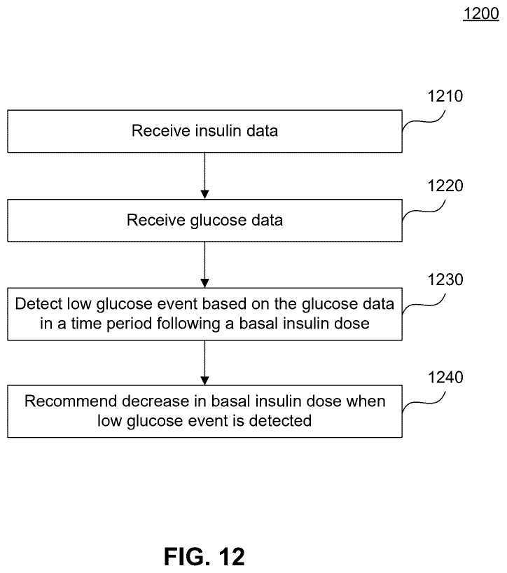

An exemplary basal insulin titration method for decreasing a basal insulin dose 1200 is shown in FIG. 12. The dose guidance system receives insulin data 1210 corresponding to basal insulin doses administered to the user. The insulin data may include a time of a basal insulin dose, an amount of the basal insulin dose, or both, among other information. The insulin data 1210 may be received by user entry or may be received from a dose detection device (e.g., smart pen cap) or from a medication delivery device (e.g., insulin pump or injection pen). The system may receive glucose data 1220. The glucose data may be received from a glucose monitoring device, such as a continuous glucose monitor. The glucose data may be retrieved from memory, or from a server or cloud.

The dose guidance system may detect a low glucose event based on the glucose data in a time period following a basal insulin dose 1230. The administration of a basal insulin dose and the time of the administration of the basal insulin dose may be determined based on the insulin data. The time period may be a fixed time period, such as at least around 20 hours, at least around 22 hours, no greater than around 30 hours and/or no greater than around 28 hours following the time of administration of the basal insulin dose. For example, the time period may be 20 hours to 30 hours, 22 hours to 28 hours, or may be around 24 hours. The time period may alternatively be defined based on a time between doses, such as the time between the basal insulin dose and a subsequent basal insulin dose.

A low glucose event may be characterized by monitored glucose levels below a low glucose level threshold for at least a minimum period of time. The low glucose event may be a severe low glucose event. For example, the low glucose event may be an event in which glucose levels are below 54 mg/dl for 15 minutes or more. However, it is understood that the low glucose event may be defined by other low glucose level thresholds or other minimum periods of time, e.g., below 60 mg/dl for 12 minutes. The low glucose event may alternatively be based on a minimum number of glucose levels below the low glucose level threshold in a predetermined period of time. The low glucose threshold for determining a low glucose event may be different than the lower glucose thresholds to which TGL is compared.

When a low glucose event is detected in the time period following the basal insulin dose, the dose guidance system may recommend decreasing the basal insulin dose 1240. The decrease may be based on an absolute amount of decrease, e.g., 1 unit, or 2 units, among other increments, or may be based on a percent decrease of the current basal insulin dose, e.g., 10%, 15% or 20%, among other increments. The dose guidance system may recommend a decrease to the basal insulin dose immediately upon detection of the low glucose event, prior to the end of the titration period. An alert may be output with the new dose amount. This helps to prevent the user from administering additional basal insulin doses at the current dose amount that resulted in the low glucose event. However, in some embodiments, the dose guidance system may determine a titration recommendation at the end of the titration period and may recommend a decrease to the basal insulin dose at the end of the titration period to ensure a sufficient amount of data is collected to make the titration determination. The decision to decrease the basal insulin dose based on the low glucose event may override a titration determination based on comparison of TGL to glucose level thresholds as described herein, for example with respect to FIG. 11.

In some embodiments described herein, the dose guidance system is configured to account for sensor bias. The sensor of the glucose monitoring device may measure glucose levels that are slightly higher or lower than the actual glucose levels in the patient's bodily fluid. Glucose sensors may have a positive bias in which the sensor reads higher than the actual blood glucose levels in the patient (by 12 mg/dl or more). The positive bias may result in the patient being titrated down to 118 mg/dl (rather than 130 mg/dl). Some glucose sensors may have a negative bias in which the sensor reads lower than the actual blood glucose levels in the patient (by 20 mg/dl or more). On average, glucose sensors are slightly negatively biased (by about 5 mg/dl).

The titration algorithm determines a titration recommendation based on comparison of a TGL to one or more glucose level thresholds as described herein. The dose guidance system may adjust one or more of the glucose level thresholds based on the sensor bias. If the sensor bias is known, the dose guidance system may adjust the upper and lower glucose level thresholds based on the known sensor bias. For example, if the sensor is known to have a 10 mg/dl positive bias, and the upper and lower glucose level thresholds are set to 80 mg/dl to 130 mg/dl, the dose guidance system may adjust the upper and lower glucose level thresholds to 90 mg/dl and 140 mg/dl, respectively, to account for the 10 mg/dl bias.

If the sensor bias is unknown, the dose guidance system may assume that on average a sensor has a slight negative bias, e.g., of 5 mg/dl. Accordingly, the dose guidance system may adjust the target range of 80 mg/dl to 130 mg/dl to 75 mg/dl to 125 mg/dl, to account for the assumed negative sensor bias of 5 mg/dl.

Dose guidance systems may recommend a dose amount of medication for a user to administer (e.g., a titrated dose recommendation). However, the dose guidance system may not directly detect the dose amount that has been administered. As a result, the dose guidance system may be unable to confirm whether the user administered the recommended dose amount, whether the user took another dose amount, or did not administer a dose at all. The dose guidance system may attempt to titrate doses to control glucose levels despite the user not administering a recommended dose. For example, if the dose guidance system recommends an increased dose, and the user does not take the newly increased dose, the user's glucose levels may remain elevated. The system may recommend further increases to the dose amount to attempt to lower glucose levels into a target range. The dose guidance system may continue to recommend increasing the next dose despite the user not administering the prior recommended dose amount. The user may ignore, not notice, or not be aware of the increases to the recommended dose. There is a risk that after several increases to the recommended dose, the user may finally notice or adhere to a new dose and administer a greatly increased dose amount. This may result in overdose which can lead to negative health effects.

Some embodiments described herein relate to a dose guidance system configured to monitor or detect user compliance, i.e., that the user is promptly taking the recommended doses, or non-compliance to reduce the risk of an overdose. Determining compliance may include determining that the user is taking the recommended amount of a dose, that the user is taking a dose at the appropriate time, or both. The system may determine compliance based on the user's interaction with the dose guidance system, based on analysis of the user's glucose data, or a combination thereof.

In some embodiments, the dose guidance system may output a recommendation for a dose of medication. The dose of medication may be an insulin dose, and may be a basal insulin dose (long-acting insulin dose). While the present disclosure may refer primarily to a basal insulin dose, the methods described herein may apply to doses of other types of insulin or to other medications. The dose recommendation may be output on a display of a component of the dose guidance system, such as a display of a display device, e.g., a smartphone or handheld receiver device, or on a display of a medication delivery device, such as a smart injection pen or dose capture device, e.g., a smart pen cap. After outputting the dose recommendation, the dose guidance system may prompt the user for an input to acknowledge the user has received or viewed the dose recommendation. The dose guidance system may determine whether an indication is received that a dose is administered within a predetermined period of time following output of the dose recommendation. For example, a medication delivery device or dose capture device of the dose guidance system may detect administration of a dose. The dose guidance system may determine compliance if an input acknowledging the dose recommendation is received, and/or if the dose is administered within the predetermined period of time that the recommended dose was viewed by the user or output to the user. The system may continue to titrate the medication dose based on the recommended dose when compliance is determined. If no input is received to acknowledge the dose recommendation, and/or if a dose is not administered within the predetermined period of time, the system may determine non-compliance. The system may take a corrective action, such as stopping titration (also referred to herein as eschewing titration) of the dose if non-compliance is determined. The system may output a notification on the display device of the user or their HCP to notify the user or HCP that titration is being stopped, i.e., that further dose guidance is being disabled. The system may allow for a predetermined number of titrations, i.e., new dose recommendations, before assessing compliance and/or taking a corrective action (e.g., stopping further titration, disabling or “turning off” the dose guidance algorithm). This is because the user may forget to acknowledge a single dose and/or may take the recommended dose late but remain generally compliant with the dose recommendations. In such situations, it would be undesirable to stop titration, which may delay reaching a suitable dose for the user and may be inconvenient for the user. However, if the patient repeatedly fails to acknowledge a new dose recommendation or to administer the recommended dose amount, then the user is more likely to be non-compliant and an intervention is appropriate.

When the user is not compliant, the dose guidance system may initiate a corrective action. The corrective action may include stopping titration of the dose. The corrective action may include outputting a notification to the user, such as to notify the user of the detection of non-compliance. The notification may advise the user that titration is being stopped and/or dose guidance is being disabled. The notification may advise the user to talk to their health care professional (HCP). The notification may include a textual explanation of why titration is being stopped. The notification may include a selectable button or icon to provide further information, such as to explain why titration is being stopped (e.g., glucose levels have not changed over a certain period of time despite the recommended dose amount increasing), and/or to explain what steps the user should take (e.g., to continue taking insulin at the most recently recommended dose amount, or to talk to their HCP). The notification may request input from the user to obtain information about the user's actions. The notification may prompt the user to confirm that the user has administered the recommended dose amount. The notification may ask the user to enter the mostly recently administered dose amount. If the user confirms that the user administered the recommended dose amount, or enters the recommended dose amount correctly, the system may resume titration of the dose. If the user indicates they are not taking the recommended dose, or enters another dose amount, the system may resume titrating the dose amount based on the amount entered by the user or based on the dose the user most recently acknowledged. The system may require input from a HCP to resume titration of the dose. For example, the HCP may enter a code on the display device of the user, or may remotely allow resuming titration by entering information at a computing device of the HCP in communication with the dose guidance system. Titration may resume at a dose amount based on a most recent dose recommendation, or at the most recently acknowledged dose by the user. Alternatively, the HCP may set the dose amount at which titration will be resumed.

When the user is non-compliant, the system may alternatively or additionally output educational information to explain the use of the dose guidance system, the importance of taking recommended dose amounts, or information related to diabetes management, among other tips or suggestions. When the user is non-compliant, the system may output a training module, text with the educational information, or links to educational materials, among other information. When a training module or educational material is output, the system may require the user to complete the training module, to view the educational material, or to confirm that the user reviewed the educational material before resuming titration of the dose.

If the user acknowledges the dose recommendation, the dose guidance system may increase a confidence level that the user is compliant and is administering the updated dose recommendation. If the user administered a dose within a predetermined time period of the dose recommendation or within a predetermined period of time of acknowledging the dose recommendation, the dose guidance system may further increase the confidence level that the user is taking the updated dose recommendation. If the user does not acknowledge the dose, the system may have a lower confidence that the user is taking the dose. If the user does not administer a dose within a predetermined time period the system may have a lower confidence that the user is compliant. The predetermined time period may be in a range of 1 minute to 30 minutes, 5 minutes to 25 minutes, or 10 minutes to 20 minutes, and may be 5 minutes, 10 minutes, or 15 minutes, among other time periods.

In some embodiments, the dose guidance system may assess the user's compliance with a dose recommendation based on glucose data collected by a glucose monitoring device of the user. The dose guidance system analyzes the glucose data to determine if the user is taking the recommended dose. If the dose guidance system recommends an increased dose amount relative to a previous dose, glucose levels are expected to be lower following the increased dose amount relative to the previous, smaller dose amount. In contrast, if the dose amount decreases, glucose levels following administration of the decreased dose amount are expected to be higher relative to the previous, larger dose amount. Thus, the dose guidance system may determine that the user is compliant with the new dose recommendation based on a glucose metric. The glucose metric may be a fasting glucose metric. The glucose metric may be a titration glucose level (TGL) as described herein.

The dose guidance system may perform a predetermined number of titrations of the dose prior to assessing compliance. For example, 2 titrations, 3 titrations, 4 titrations, or any number of predetermined titrations. If a change in the glucose metric is not determined after the predetermined number of titrations, or the change is less than a predetermined amount, then the user is assessed to be non-compliant with the dose recommendations and the system takes a corrective action, such as stopping further titration of the dose and preventing output of new dose recommendations.

If no change in the glucose metric is detected or the change is less than a predetermined threshold, the dose guidance system is configured to perform a corrective action. The corrective action may include outputting on a display device, a request for user confirmation of the dose amount to be administered. The system may prompt the user to enter or select a numerical value of the dose amount that was most recently administered (e.g., “Please enter the amount of your most recently administered dose”). Alternatively, the system may prompt the user to confirm that they took a dose corresponding to the recommendation (e.g., “Did you administer a dose of 10 U?”). The corrective action may include stopping or eschewing further titration of the dose such that no further new dose recommendations are output. In such embodiments, the system may output a notification to the user that titration is stopped. The system may alternatively or additionally output a notification to an HCP or caregiver of the user.

In some embodiments, the corrective action may include changing a titration period. Each new dose recommendation may be determined over a first titration period, such as 3 days, and if the system determines that the user is non-compliant, the system may perform subsequent titrations based on a second titration period, that is longer than the first titration period, such as 5 days. Increasing the titration period may allow for more days of data to be collected to determine if the user is taking the recommended dose or if the dose is having the intended effect. Further, the longer titration period results in the dose changing more gradually, providing the user with more time and opportunity to notice and begin taking a new dose.

In some embodiments, the corrective action may include increasing a frequency at which the system prompts the user to confirm the dose amount that was administered. The system may prompt the user to confirm a dose administered at a first frequency. The first frequency may be once every two days, once every three days, once per week, among other intervals. The frequency may be increased to a second frequency when non-compliance is detected. For example, when a number of new dose recommendations that have been output exceeds a threshold number with no change in the glucose metric detected, or with the change in the glucose metric being below a threshold change, the system may increase the frequency at which the user is prompted to confirm administration of the recommended dose. In some examples, the system may only prompt the user to confirm a dose amount administered at a first frequency following a titration (e.g., starting after a new dose amount is recommended).

In some embodiments, the dose guidance system may assess a confidence level that the user is compliant. For example, if the user acknowledges that they viewed a dose recommendation, administers a dose within a predetermined time period of the dose recommendation, and a change in glucose metric is determined, then the system may have a very high confidence level that the user is compliant with the dose recommendation. However, in a second example, if the user has not acknowledged dose recommendations or has not administered a dose within the predetermined time of viewing the dose recommendation, and the system does not detect a change in the glucose metric, then the system may have a very low confidence that the user is complying with the updated dose recommendation and/or may determine that the user is not complying with the dose recommendation. The system may require the confidence level to be at or above a predetermined confidence level. If the confidence level is below the predetermined confidence level, a corrective action may be taken as discussed herein. If the confidence level is at or above the predetermined confidence level, the dose recommendation system may continue to titrate the dose.

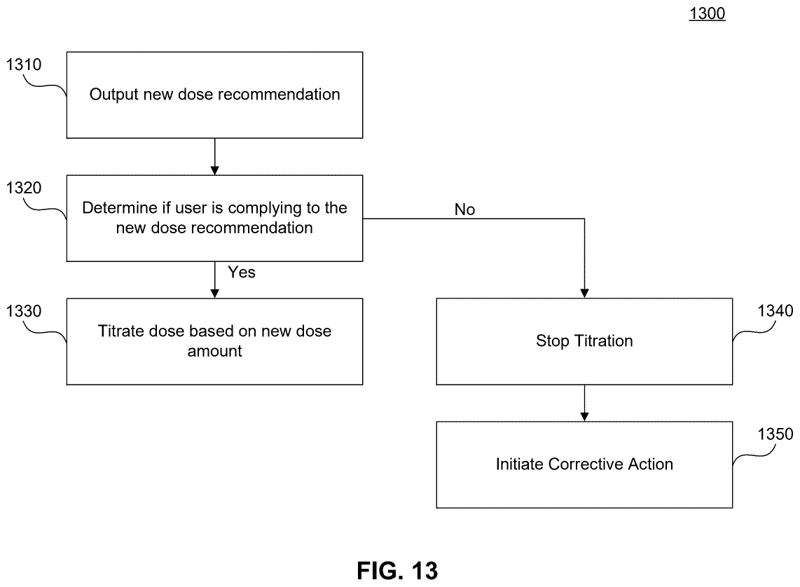

An exemplary method of determining user compliance with dose recommendations 1300 based on user interaction with a dose guidance system is shown in FIG. 13. The dose guidance system outputs a new dose recommendation 1310 (e.g., a new dose amount). The system determines if the user is complying with the dose recommendation 1320. If the system determines the user is complying with the dose recommendation, the system titrates the dose based on the new dose recommendation 1330. If the system determines the user is not compliant, the system may stop or disable titration 1340 and not output any further new dose recommendations. The system may output a notification indicating that no further dose recommendations will be provided, i.e., that the dose guidance is stopped or disabled. Further, if the user is not compliant, the system may initiate a corrective action 1350. The corrective action may aim to understand the dose taken by the user and/or to educate the user to encourage compliance with the dose recommendations.

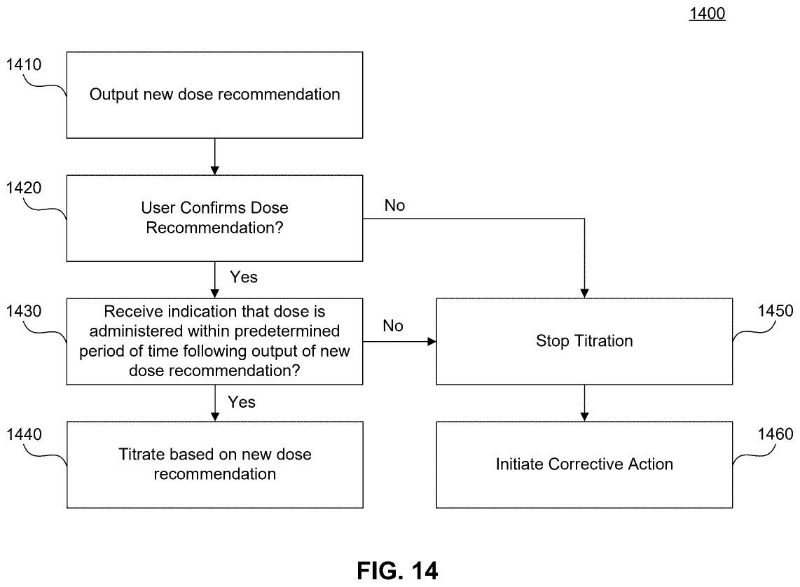

An exemplary method of determining compliance with dose recommendations 1400 based on user interaction with a dose guidance system is shown in FIG. 14. A new dose recommendation is output 1410. The system checks user confirmation of the new dose recommendation 1420 (e.g., a new dose amount). The system may confirm that the user viewed the new dose recommendation, or may prompt the user to confirm they reviewed and received the new dose recommendation. The system may alternatively or additionally receive an indication that a dose is administered within a predetermined period of time following output or acknowledgement of the new dose recommendation 1430. The system may determine the user is complying with the new dose recommendation if the input is received to acknowledge the dose recommendation and/or if the dose is administered within the predetermined period of time following output of the dose recommendation. The system may continue to titrate the dose and provide new dose recommendations 1440 when the user is determined to be compliant. Alternatively, if the user does not acknowledge the dose recommendation and/or if the user does not administer the dose within a predetermined period of time following output of the new dose notification, the system may determine the user is non-compliant. The system may stop titration 1450 and disable the dose guidance from providing further new dose recommendations. The system may output a notification on a display device of the user, HCP, or both. The system may initiate a corrective action 1460 as described herein.

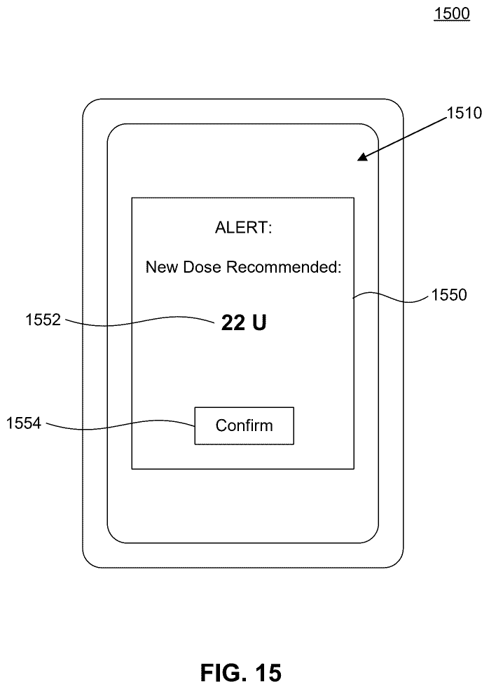

As discussed above, when determining compliance, the dose guidance system may prompt the user to confirm or acknowledge the new dose recommendation. The acknowledgement may include receipt of a user input at a user interface of a pen cap, a mobile phone, or other component of the dose guidance system. As shown for example in FIG. 15, a mobile device 1500 may include a user interface 1510. An alert or notification 1550 may be displayed on user interface 1510. Alert 1550 may include a new dose recommendation 1552. Alert 1550 may include the new dose recommendation 1552 and may optionally include the previous dose amount, and/or the amount of change in the dose. Alert 1550 may include a button or icon 1554 for the user to confirm or acknowledge the new dose recommendation. The system may determine that the user is compliant with the new dose recommendation if the input is received. While FIG. 15 shows an alert presented on a mobile device, it is understood that the alert or notification may be displayed on a user interface of a pen cap, medication delivery device, or other component of the dose guidance system.

Dose guidance system may receive an indication that the dose is administered. Dose guidance system may include a medication delivery device or dose capture device configured to automatically record and communicate administration of a dose by the medication delivery device. The medication delivery device may include, for example, an infusion pump, a patch pump, or a smart injection pen. The dose capture device may include, for example, a smart pen cap removably securable to an injection pen. A pen cap may be configured to detect when the pen cap is uncapped from or capped onto a manual medication delivery device, such as an injection pen. The pen cap may infer that a dose is administered based on uncapping the pen cap from the injection pen, capping the pen cap onto the injection pen, or based on a sequence of uncapping and subsequent capping of the pen cap onto the injection pen. The pen cap may communicate the dosing event to other components of dose guidance system, such as to a display device. In some embodiments, the medication delivery device may record dosing events and communicate the dosing events to other components of the dose guidance system. The medication delivery device may include a user interface configured to present alerts or notifications regarding new dose recommendations and to receive user confirmation as described herein.

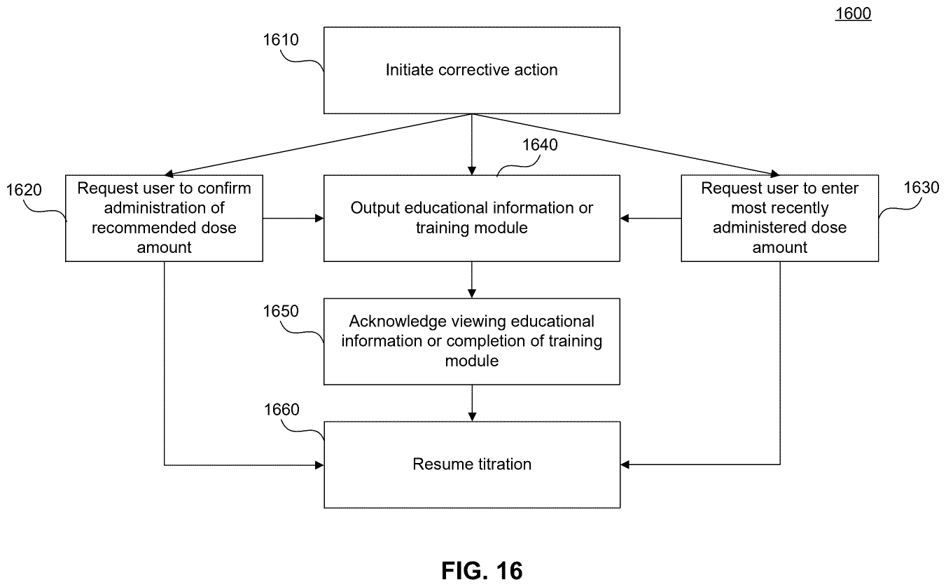

An exemplary method 1600 for initiating a corrective action is shown in FIG. 16. Dose guidance system may initiate a corrective action 1610 when the user is determined to be non-compliant with the dose recommendation as described herein. The system may request the user to confirm that the user has administered the recommended dose amount 1620. If the user confirms, such as by entering an input on a user interface, the system may resume titration 1660 based on the recommended dose amount. If the user does not confirm that they administered the recommended dose amount, the system may output educational information or a training module 1640 in order to encourage the user to resume complying with the dose recommendations. The system may resume titration 1660 after outputting the educational information or training module. Optionally, the system may require acknowledgement that the user viewed the educational information or that the user completed the training module 1650 before resuming titration 1660. The system may require the user to enter an input, such as by selecting a button or icon, to indicate that they reviewed the materials. The corrective action may alternatively include outputting a request for the user enter the most recently administered dose amount 1630. If the entered value is the same as the recommended dose amount, the system may resume titration 1660. If the entered value is not the same as the recommended dose amount, the system may output educational information or a training module 1640 as discussed. Alternatively, the system may initiate a corrective action 1610 and output educational information or training module 1640 without requesting the user to confirm the dose amount.

Some embodiments described herein relate to determining user compliance with a dose recommendation based on glucose data. Glucose data may be collected by a glucose monitoring device worn by the user, as discussed herein. The glucose data collected following output of a dose recommendation may be analyzed to determine if the user is compliant with the recommended dose. Determining compliance with dose recommendations based on glucose data is beneficial as no user input is required which may improve the user experience. Further, the glucose data may be more reliable than inferring the user's dosing based on interaction with the user interface. The user may incorrectly enter an administered dose into the user interface or may agree that they are taking the new dose despite not taking the new dose.

In some embodiments, the dose guidance system may titrate an insulin dose by determining and outputting a new dose recommendation at an end of each of a plurality of titration periods based on glucose data received during the titration period. The titration period may include two or more days. The system may receive an indication that a user administers a basal insulin dose each day of the titration period, such as by detecting capping and uncapping of a smart pen cap on an insulin injection pen. The system determines a glucose metric, such as a TGL, for each day of the titration period. The system determines user compliance with the dose recommendations based on the change in the glucose metric, e.g., TGL over time. If the new dose recommendations are increasing over the plurality of titration periods (e.g., 30 units to 33 units to 36 units), compliance is determined if the TGL is decreasing over time. The decrease in TGL may be determined based on a slope of a line on a plot of TGL-dose values. In contrast, if the dose recommendations are decreasing in each titration, compliance is determined based on an increase in the TGL over time. In some embodiments, the change in TGL can be compared to an expected changed in TGL to determine compliance, and the system may determine if the actual change in TGL is within a predetermined amount of an expected change in TGL. The expected change in TGL may be determined based on the user's insulin sensitivity.

While the present application primarily refers to the use of TGL, other glucose metrics may be used, such as other fasting glucose level metrics.

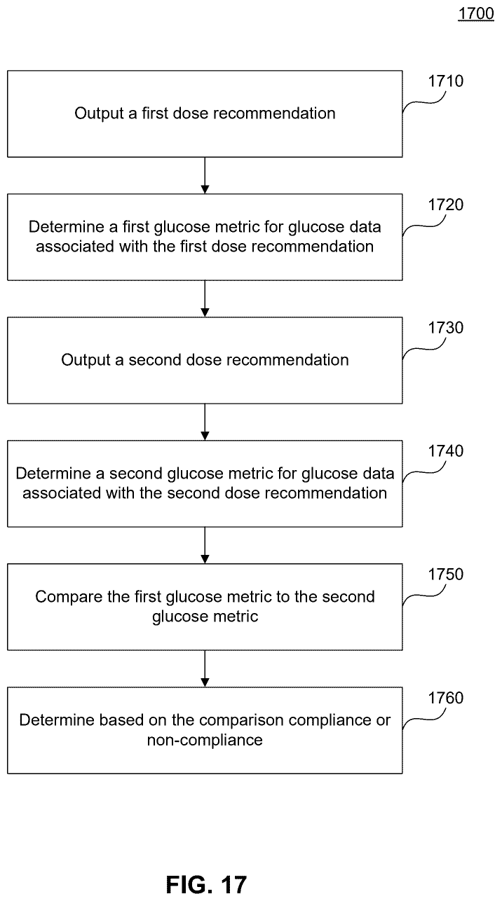

An exemplary method for determining compliance with dose recommendations based on collected glucose data is shown in FIG. 17. Dose guidance system may output a first dose recommendation 1710. Dose guidance system may determine a first glucose metric based on glucose data received after the first dose recommendation 1720. A second dose recommendation may be output 1730 after a first titration period. The first titration period may be 2 or more days, 3 or more days, or may be in a range of 2 to 10 days, 2 to 7 days, 3 to 7 days, or less than 10 days. A second glucose metric is determined based on glucose data received after output of the second dose recommendation 1740. The first glucose metric is compared to the second glucose metric 1750. The system may determine compliance to the dose recommendations based on the comparison of the first glucose metric and the second glucose metric 1760.

In some embodiments, the glucose metric may be indicative of a fasting glucose level. The glucose metric may be a TGL as described herein, among other glucose metrics. The glucose metric may be determined for each dose administered by the user, such as based on glucose data collected in a period of time following output of the dose recommendations or following administration of the dose (e.g., glucose data collected at the time the dose is administered up to the time of a subsequent dose). The system may determine and store pairs of dose-TGL values.

The dose guidance system may determine a relationship between the dose and the TGL (or other glucose metric). The dose guidance system may determine whether the direction of change in the glucose metric is consistent with the change to the recommended dose. As the amount of the dose increases, the TGL should decrease, and conversely as the amount of the dose decreases, the TGL should increase. The slope of TGL to dose may represent the user's insulin sensitivity factor (ISF). If the slope is 0, then the TGL is not changing despite the change in dose recommendation. As a result, the dose guidance system may determine that when the slope is 0 or near 0, the user is not complying with the new dose recommendation. If the recommended dose is increasing and the slope is negative, the dose guidance system may determine that the user is complying with the new dose recommendation. If the slope is positive, representing an increased TGL, despite an increased dose recommendation, the dose guidance system may determine non-compliance.

In some embodiments, the dose guidance system may be configured to complete a predetermined number of titrations, i.e., to adjust the insulin dose a predetermined number of times before assessing compliance or non-compliance. The predetermined number titrations may be in a range of 2 to 5 titrations, or 3 to 4 titrations. This is because one or two dose increases are unlikely to cause hypoglycemia if the user misses the first increased dose recommendation and begins administering the second increased dose recommendation. For example, if the initial dose is 10 U, a first increased dose recommendation is 12 U, and a second increased dose recommendation is 14 U, the user is unlikely to experience hypoglycemia if the user misses the 12 U dose and begins administering a 14 U dose. However, if the user fails to notice several dose increases after several titrations, and suddenly begins administering a 20 U dose, the risk of hypoglycemia upon administering the new dose is much greater.

Further, glucose metrics, such as TGL, may change based on a variety of other factors other than user compliance with dose recommendations. For example, glucose metrics such as TGL may vary with changes in diet (e.g., consuming more or fewer carbohydrates), changes in exercise, changes in stress levels, changes in medications (e.g., GLP-1), changes in other medications (e.g., steroids), sickness or other conditions or comorbidities, among other factors. Thus, the user may be compliant but a corresponding change in TGL may not be evident after a single titration (i.e., a single change in dose). As each titration may be based on glucose data collected over a predetermined period, for example, 2 days to 3 days, there may be relatively little data collected on which to determine compliance. Waiting a longer period of time, e.g., multiple titrations, allows for collection of additional data to more accurately assess whether the user's TGL is changing over time in response to increases to the dose.

In a further example, if the user's initial dose is 20 U, a first titration recommends a dose of 23 U after 3 days, the user may be complying with the new dose, but the TGL may show no change over 3 days due to other factors that impact glucose levels, and the relatively small change in the dose of 3 U. If, however, a second titration recommends a dose of 26 U after 3 more days, a larger amount of data is collected (6 days total), and the change in dose relative to the initial dose is greater (6 U) which would be expected to result in a correspondingly greater glucose response. Thus, after two titrations, the system has more data on which to determine a change in TGL and thus more confidence in the determination of compliance. If a third titration after 3 more days recommends an increased dose of 29 U, the system would have greater confidence in the assessment of a change in TGL, as a 9 U change would be expected to elicit a greater glucose response. However, the 29 U dose is nearly 50% greater than the initial dose 20 U, and if the user had not been complying with the series of increasing dose recommendations (e.g., 20 U, 23 U, 26 U), taking the recommended dose of 29 U may be more likely to result in an overdose of insulin. Thus, waiting too long before determining compliance may allow for too large of an increase in the dose, which may result in potential overdosing of insulin if the user suddenly complies with the increased dose recommendation.

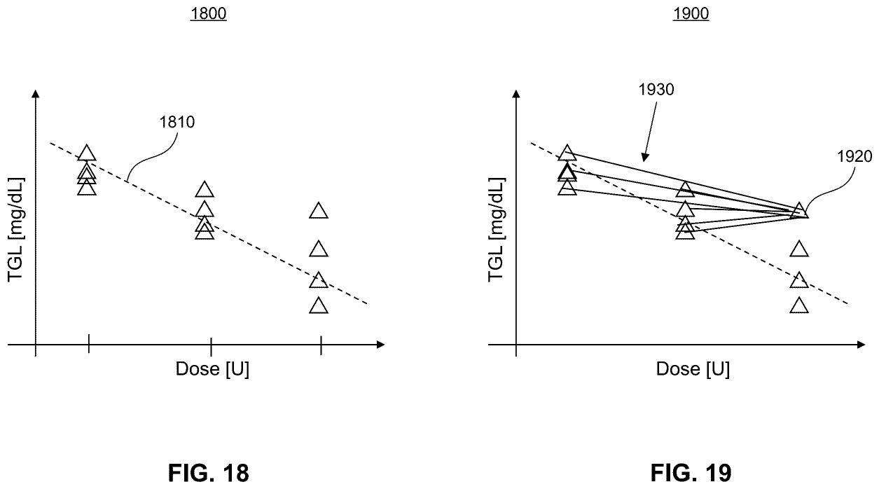

FIG. 18 shows an exemplary plot of TGL values for each administered dose 1800. FIG. 18 shows four doses administered at a first dose amount, four doses administered at a second dose amount, and four doses administered at a third dose amount. A fit line 1810 may be generated for the TGL-dose values to determine a relationship between TGL and dose. A Theil-Sen estimator may be used to fit a line to sample points by selecting a median of the slope lines through the pairs of data points. The use of the Theil-Sen estimator may be advantageous over ordinary least squares in that it is more insensitive to outliers. This can help to account for variations in TGL that may be the result of lifestyle factors. However, it is understood that other methods of determining a fit line known in the art may be used. The slope of the fit line on the plot of TGL versus dose corresponds to the patient's insulin sensitivity factor (ISF). The system may store a range of acceptable ISF values. The range of ISF values may be based on population data and/or a physiologically acceptable range of values. When the determined ISF is within the range of acceptable ISF values, the system determines that the user is compliant with the dose recommendations and can continue to titrate the insulin dose.

If the slope of the fit line 1810 is positive or zero slope, the system determines that the user is non-compliant with the dose recommendations (assuming dose recommendations are successively increasing). If the median slope is negative, the system determines that the user is compliant.

In some embodiments, if the median slope is negative, the system may perform a further check to determine compliance and to ensure that it is safe to continue titration of the insulin dose. The system reviews the distribution of all computed slopes. If the user is taking the recommended dose (assuming the doses are increasing over time), there should be relatively few slopes that are positive.

As shown for example in FIG. 19, for each new TGL-dose pair 1920, slopes 1930 can be calculated for the current TGL-dose values 1920 relative to all previous TGLs at different doses. If the percentage of positive slopes of all slopes is below a threshold percentage, the system determines that the user is compliant with the dose recommendations. In some embodiments, the threshold may be 30%, 25%, or 20%. If the percent of positive slopes is at or above the threshold, the system may proceed to determine a risk of sudden compliance.

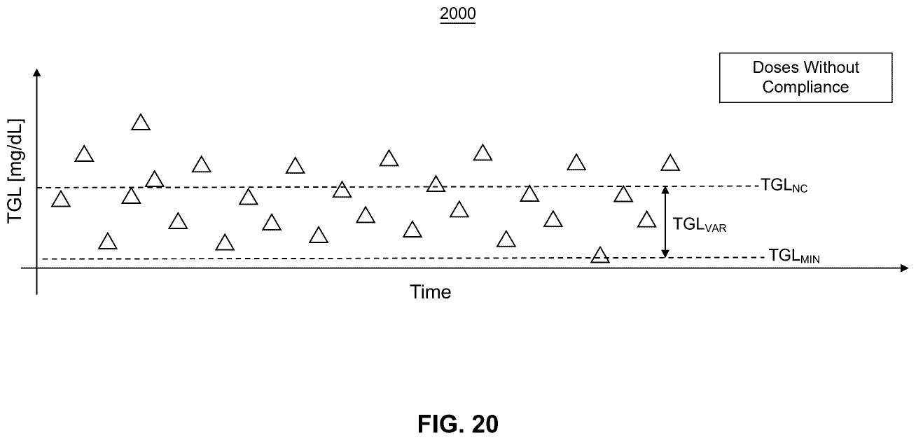

For each dose recommendations, the system may determine the risk of whether sudden compliance with the dose recommendation would result in dangerously low glucose levels. As shown in FIG. 20, a plot 2000 may be constructed based on the TGL for each administered dose. The median TGL, referred to as TGL_{NC}, may be determined based on each of the TGL values. A minimum TGL value, i.e., the lowest of all determined TGL values for the user, referred to as TGL_{MIN}, is determined based on the TGL values. A TGL variability TGL_{VAR} is determined based on a difference between the median TGL value and the minimum TGL values. The TGL variability may represent the day-to-day change in TGL at a fixed dose. High TGL variability increases the risk of hypoglycemia at a given TGL.

The risk of sudden compliance is low if expression (1) is true:

$${D}_{next} < \frac{{TGL}_{MIN} - K}{{β}_{max}} + {D}_{NC}$$

wherein Dₙₑₓₜ is the recommended dose, TGLₘᵢₙ is the minimum TGL of the user's TGLs, K is a predetermined value (e.g., 54), and βₘₐₓ is a maximum insulin sensitivity factor (ISF), and D_{NC} is the most recently acknowledged dose taken by the user. K may be 54 mg/dl which is the threshold for very low or urgent low glucose. However, if the user has a fear of hypoglycemia, K may be set to a higher value, such as 70 mg/dl. K may be selected based on risk and can be in a range of 54 mg/dl to 70 mg/dl, and the higher the value of K the more conservative the approach.

The risk may be considered to be very low if expression (2) is true:

$${D}_{next} < \frac{{TGL}_{min} - {K}_{2}}{{β}_{max}} + {D}_{NC}$$

wherein Dₙₑₓₜ is the new dose recommendation, TGLₘᵢₙ is the minimum TGL of the user's TGLs, K₂ is a predetermined constant (e.g., 70), wherein K₂ is greater than K, βₘₐₓ is the maximum insulin sensitivity factor (ISF), and D_{NC} is the most recently acknowledged dose taken by the user. K₂ may be other values and may be in a range of 70 mg/dl to 90 mg/dl. K₂ may be selected based on the patient's risk tolerance. As discussed with respect to K, higher values of K₂ correspond to a more conservative approach.

The system may continue to titrate the insulin dose when the risk of sudden compliance is assessed to be low or very low, such as based on expression (1) or (2).

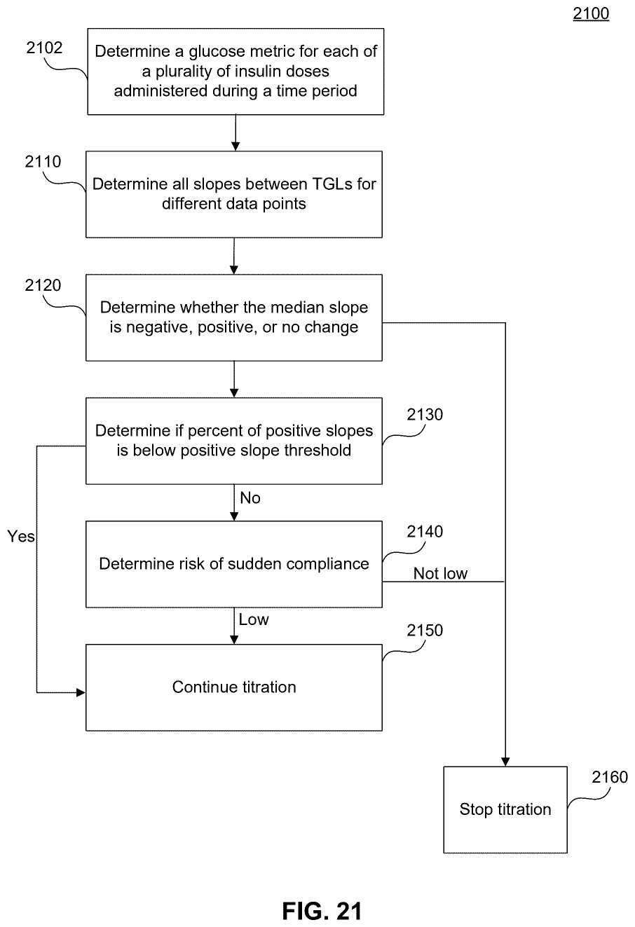

An exemplary method for determining compliance with dose recommendations based on glucose data and accounting for risk of sudden compliance 2100 is shown in FIG. 21. The system may determine a glucose metric, such as a TGL, for each insulin dose administered during a time period 2102. For each new dose recommendation, the system determines all slopes between the current dose and TGL and all TGLs for previous doses 2110. The system determines whether a median slope of all slopes is negative 2120. If the median slope is not negative (e.g., is positive or zero slope), further titration of the dose is stopped or eschewed 2160 as the user may be non-compliant (assuming the dose recommendations are increasing over time). If the median slope is negative, the system may determine the user is compliant and continues to titrate the insulin dose. Optionally, if the median slope is negative, the system may further determine if a percent of slopes that is positive of all slopes is below a positive slope threshold 2130. If the percent of slopes that is positive is below the positive slope threshold, the system continues titration of the dose 2150. If the percent of slopes that is positive is above the positive slope threshold, the system further determines a risk of sudden compliance 2140. The risk of sudden compliance may be computed as discussed herein (e.g., based on expression 1 or 2) and determines whether the user administering the recommended dose would likely result in hypoglycemia. If the risk of sudden compliance is low, the system may continue to titrate the dose 2150. If the risk of sudden compliance is not low, the system will stop titration of the dose 2160. The system may output a notification to a display device of the user or a computing device of the HCP and/or initiate a corrective action as described herein. Dose guidance and titration of the dose may be resumed after the corrective action is taken. Dose guidance may be resumed based on an input received at the display device from an HCP, such as entering a code, or by receiving input from a computing device of the HCP in communication with the dose guidance system.

In some embodiments, dose guidance system may determine an expected change in the glucose metric and may determine compliance based on comparison of the actual glucose metric to the expected glucose metric. The expected glucose metric may be a range of values. If the relationship of the change in TGL to change in dose is known (i.e., the slope of fit line 1910 in FIG. 18), then the system can predict the TGL for a given dose amount. The system may determine a TGL variability based on the difference in TGL values at a given dose (as shown for example in FIG. 20). The TGL variability may be used to construct a range of expected TGL values at a given dose. The system may determine TGL in a titration period following a dose recommendation and determine if the TGL is within the expected range of TGL values. If so, the system may determine the user is compliant with the dose recommendations. Otherwise, if the TGL is not within the expected range of TGL values, the system may determine the user is non-compliant with the dose recommendations.

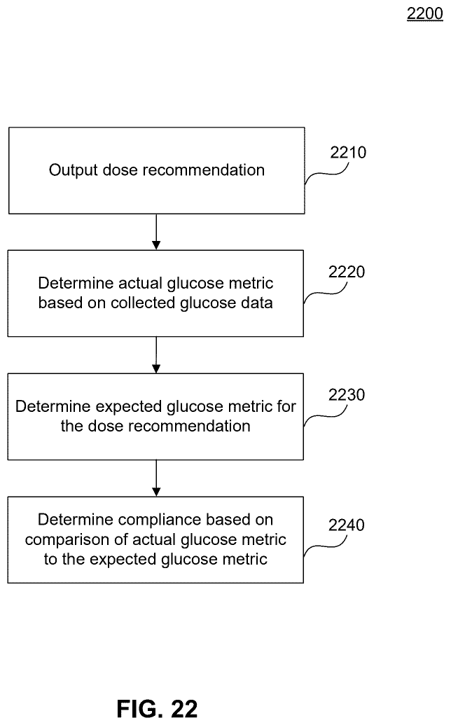

An exemplary method of determining compliance with a dose recommendation based on an expected glucose metric is shown in FIG. 22. The system may output a dose recommendation 2210. The system may determine a glucose metric based on glucose data collected following the dose recommendation 2220. The glucose metric may be, for example, TGL. The system may determine an expected glucose metric for the dose recommendation 2230. The expected glucose metric may be based on a known relationship between the dose and the glucose metric. The expected glucose metric may be based on an insulin sensitivity factor. The system determines compliance based on comparison of the glucose metric to the expected glucose metric 2240. The system may determine compliance if the glucose metric is the same as or within a predetermined amount of the expected glucose metric.

Alternatively, the system may determine a range for the expected glucose metric based on the expected glucose metric and a glucose metric variability. The glucose metric variability may be computed based on recorded values of the glucose metric at a given dose. The glucose metric variability may be a difference between a median glucose metric and a minimum value of the glucose metric. The system determines compliance if the actual glucose metric is within the determined range for the expected glucose metric.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the claims and their equivalents.

The disclosure of this application also contains the following numbered clauses:

Clause 1. A system for titrating a basal insulin dose, the system comprising:
a glucose monitoring device comprising sensor electronics coupled to an in vivo glucose sensor configured to collect glucose data of a patient;
one or more processors in communication with the glucose monitoring device; and
a memory coupled to the one or more processors and storing instructions that when executed by the one or more processors cause the one or more processors to:
   determine an initial basal insulin dose,
   receive insulin data during a titration period, wherein the titration period comprises a plurality of days,
   receive glucose data for each day of the plurality of days in the titration period,
   assess a validity of each of the plurality of days in the titration period,
   determine a titration glucose level for each day in the titration period that is determined to be valid,
   compare the titration glucose level for each day in the titration period that is determined to be valid to one or more glucose level thresholds, and
   output a recommended adjustment to the basal insulin dose based on the comparison.

Clause 2. A system for titrating a basal insulin dose, the system comprising:
one or more processors in communication with a glucose monitoring device; and
a memory coupled to the one or more processors and storing instructions that when executed by the one or more processors cause the one or more processors to:
   determine an initial basal insulin dose,
   receive insulin data during a titration period, wherein the titration period comprises a plurality of days,
   receive glucose data of a patient collected by the glucose monitoring device for each day of the plurality of days in the titration period,
   determine a titration glucose level for each day in the titration period,
   compare the titration glucose level for each day in the titration period to one or more glucose level thresholds, and
   output a recommended adjustment to the basal insulin dose based on the comparison.

Clause 3. The system of clause 2, further comprising a glucose monitoring device configured to collect the glucose data.

Clause 4. The system of clause 2 or 3, wherein the one or more processors are further caused to assess a validity of each of the plurality of days in the titration period, determine a titration glucose level for each day in the titration period that is determined to be valid, and compare the titration glucose level for each day in the titration period that is determined to be valid to the one or more glucose level thresholds.

Clause 5. The system of any of clauses 1 to 4, wherein the titration glucose level is determined by dividing each day into a plurality of time blocks, and calculating an average glucose level for each of the plurality of time blocks, wherein the titration glucose level is a lowest average glucose level of the plurality of time blocks.

Clause 6. The system of any of clauses 1 to 5, wherein each day of the plurality of days comprises a time period between a first basal insulin dose and a second basal insulin dose.

Clause 7. The system of clause 6 when dependent on clause 1 or 4, wherein the day is assessed to be valid when the time period is greater than or equal to a minimum duration.

Clause 8. The system of clause 6 or 7 when dependent on clause 1 or 4, wherein the day is assessed to be valid when the time period is less than or equal to a maximum duration.

Clause 9. The system of any of clauses 6 to 8, wherein the day is assessed to be invalid when the time period is less than the minimum duration.

Clause 10. The system of any of clauses 6 to 8, wherein the day is assessed to be invalid when the time period is greater than a maximum duration.

Clause 11. The system of any of clauses 1 to 10, wherein the recommended adjustment is to decrease the basal insulin dose when a number of days for which the titration glucose level is below a lower glucose level threshold of the one or more glucose level thresholds meets or exceeds a minimum down-titration period threshold.

Clause 12. The system of any of clauses 1 to 11, wherein the recommended adjustment is to increase the basal insulin dose by a first amount when a number of days for which the titration glucose level meets or exceeds a first upper glucose level threshold of the one or more glucose level thresholds meets or exceeds a minimum up-titration period threshold.

Clause 13. The system of clause 12, wherein the recommended adjustment is to increase the basal insulin dose by a second amount when a number of days in which the titration glucose level exceeds a second upper glucose level threshold of the one or more glucose level thresholds meets or exceeds the minimum up-titration period threshold, wherein the second upper glucose level threshold is greater than the first upper glucose level threshold, and wherein the second amount is greater than the first amount.

Clause 14. The system of any of clauses 1 to 13, wherein the recommended adjustment to the basal insulin dose is output at an end of the titration period and without user input.

Clause 15. The system of any of clauses 1 to 14, wherein the initial basal insulin dose is determined based on a body weight of the user.

Clause 16. The system of any of clauses 1 to 15, wherein the one or more processors are further caused to detect a low glucose event in a period of time following administration of a basal insulin dose, and the recommended adjustment is to decrease the basal insulin dose based on detection of the low glucose event.

Clause 17. The system of clause 16, wherein the low glucose event comprises glucose levels of the user are below a low glucose level threshold for at least a minimum period of time.

Clause 18. The system of any of clauses 1 to 17, wherein the one or more processors are configured to determine overbasalization when a ratio of the basal insulin dose to a body weight of the user meets or exceeds a dose to body weight ratio threshold, and when overbasalization is determined, the one or more processors are configured to stop output of recommendations to increase the basal insulin dose.

Clause 19. The system of clause 1 or 3 or any clause dependent thereon, wherein at least one of the one or more glucose level thresholds is adjusted based on a sensor bias of the glucose monitoring device.

Clause 20. A method for titrating a basal insulin dose, the method comprising: determining, by one or more processors, an initial basal insulin dose, receiving, by the one or more processors, insulin data during a titration period, wherein the titration period comprises a plurality of days,
receiving, by the one or more processors, glucose data for each day of the plurality of days in the titration period, wherein the glucose data is received from a glucose monitoring device comprising sensor electronics coupled to an in vivo glucose sensor,
assessing, by the one or more processors, a validity of each of the plurality of days in the titration period,
determining, by the one or more processors, a titration glucose level for each day in the titration period that is determined to be valid,
comparing, by the one or more processors, the titration glucose level for each day in the titration period that is determined to be valid to one or more glucose level thresholds, and
outputting, by the one or more processors, a recommended adjustment to the basal insulin dose based on the comparison.

Clause 21. A method for titrating a basal insulin dose, the method comprising:
determining, by one or more processors, an initial basal insulin dose,
receiving, by the one or more processors, insulin data during a titration period, wherein the titration period comprises a plurality of days,
receiving, by the one or more processors, glucose data for each day of the plurality of days in the titration period,
determining, by the one or more processors, a titration glucose level for each day in the titration period,
comparing, by the one or more processors, the titration glucose level for each day in the titration period to one or more glucose level thresholds, and
outputting, by the one or more processors, a recommended adjustment to the basal insulin dose based on the comparison.

Clause 22. The method of clause 20 or 21, further comprising determining the titration glucose level by dividing each day into a plurality of time blocks, and calculating an average glucose level for each of the plurality of time blocks, wherein the titration glucose level is a lowest average glucose level of the plurality of time blocks.

Clause 23. The method of any of clauses 20 to 22, wherein the validity of each of the plurality of days is assessed based on a comparison of a duration of each day to a minimum duration and a maximum duration.

Clause 24. The method of any of clauses 20 to 23, wherein the recommended adjustment is to decrease the basal insulin dose when a number of days in which the titration glucose level is below a lower glucose level threshold of the one or more glucose level thresholds meets or exceeds a minimum down-titration period threshold.

Clause 25. The method of any of clauses 20 to 24, wherein the recommended adjustment is to increase the basal insulin dose by a first amount when a number of days in which the titration glucose level meets or exceeds an upper glucose level threshold of the one or more glucose level thresholds meets or exceeds a minimum up-titration period threshold.

Clause 26. The method of any of clauses 20 to 25, further comprising detecting a low glucose event in a period of time following administration of a basal insulin dose, and recommending an adjustment to decrease the basal insulin dose based on detection of the low glucose event.

Clause 27. The method of any of clauses 20 to 26, further comprising detecting overbasalization when a ratio of the basal insulin dose to a body weight of the user meets or exceeds a dose to body weight ratio threshold, and when overbasalization is detected, stopping output of recommendations to increase the basal insulin dose.

Clause 28. An apparatus comprising means for performing the method of any of clauses 20 to 27.

Clause 29. A computer program, computer program product or computer readable medium comprising software code adapted, when executed by a computer system, to perform a method as set out in any of clauses 20 to 27.

Clause 30. A method for detecting compliance with insulin dose titration recommendations provided by a dose guidance system, the method comprising:
outputting, on a display of the dose guidance system, a new dose recommendation for an insulin dose;
receiving, by a processor in communication with the display, user confirmation of the new dose recommendation;
receiving, by the processor, an indication that a dose was administered within a predetermined period of time after output of the new dose recommendation; and
titrating, by the processor, the insulin dose when user confirmation is received and when the indication that the dose was administered within the predetermined period of time is received; and
eschewing titration of the insulin dose when the user confirmation is not received or when the indication that the dose was administered is not received within the predetermined period of time after output of the new dose recommendation.

Clause 31. The method of clause 30, wherein receiving user confirmation of the new dose recommendation comprises receiving a user input to acknowledge the user viewed the new dose recommendation.

Clause 32. The method of clause 30 or 31, wherein receiving the indication comprises detecting administration of the dose by a dose capture device or by a medication delivery device of the dose guidance system.

Clause 33. The method of any of clauses 30 to 32, further comprising initiating a corrective action when the user confirmation is not received or when the indication that the dose was administered is not received within the predetermined period of time after output of the new dose recommendation.

Clause 34. The method of clause 33, wherein the corrective action comprising outputting a prompt for the user to enter a most recently administered dose amount or outputting a prompt for a user to confirm administration of the new dose recommendation.

Clause 35. The method of clause 34, wherein the method further comprises resuming titration of the insulin dose when the most recently administered dose amount entered by the user is the same as the new dose recommendation or when the user confirms administration of the new dose recommendation.

Clause 36. The method of any of clauses 33 to 35, wherein the corrective action comprises outputting educational information.

Clause 37. The method of clause 30, wherein titrating the insulin dose comprises outputting a second new dose recommendation on the display of the dose guidance system.

Clause 38. A method for detecting compliance with insulin dose recommendations provided by a dose guidance system, the method comprising:
outputting a first dose recommendation for an insulin dose on a display of the dose guidance system;
determining, by a processor of the dose guidance system, a first glucose metric based on glucose data received from a glucose monitoring device after output of the first dose recommendation;
outputting, by the processor, a second dose recommendation for the insulin dose on the display of the dose guidance system based on the glucose data received after output of the first dose recommendation;
determining, by the processor, a second glucose metric based on the glucose data received after output of the second dose recommendation;
comparing, by the processor, the first glucose metric to the second glucose metric;
determining, by the processor, compliance or non-compliance based on the comparison of the first glucose metric to the second glucose metric;
eschewing titration of the insulin dose by the processor when compliance is not determined; and
outputting a notification on the display indicating that further titration of the insulin dose is disabled.

Clause 39. The method of clause 38, wherein the insulin dose is a basal insulin dose.

Clause 40. The method of clause 38 or 39, wherein the first glucose metric is a titration glucose level.

Clause 41. The method of any of clauses 38 to 40, wherein the second dose recommendation is greater than the first dose recommendation, and wherein determining compliance or non-compliance comprises determining that the second glucose metric is lower than the first glucose metric.

Clause 42. The method of any of clauses 38 to 41, further comprising titrating the insulin dose when compliance is determined.

Clause 43. A system for determining user compliance with insulin dose recommendations, the system comprising:
a glucose monitoring device, comprising:
a glucose sensor comprising a first portion configured to be positioned above a skin surface of a user and a second portion configured to be positioned below a skin surface and in contact with a bodily fluid of the user for monitoring glucose levels in the bodily fluid, and
sensor electronics coupled to the first portion of the glucose sensor and comprising communication circuitry for transmitting glucose data by a wireless communication protocol; and
one or more processors coupled to a memory storing instructions that, when executed by the one or more processors, cause the one or more processors to:
   determine a glucose metric based on the glucose data associated with each of a plurality of insulin doses administered during a time period;
   determine a plurality of slopes between a current insulin dose and a current glucose metric to each of the other insulin doses and the corresponding glucose metrics;
   determine a median slope of the plurality of slopes;
   determine compliance with dose recommendations based on the median slope of the plurality of slopes; and
   eschew titration of the insulin dose when compliance is not determined.

Clause 44. The system of clause 43, wherein the glucose metric is a titration glucose level.

Clause 45. The system of clause 43, wherein the one or more processors are further caused to determine compliance when the median slope is negative and when the dose recommendations increased over the time period.

Clause 46. The system of clause 43, wherein the one or more processors are further caused to titrate the insulin dose when compliance is determined.

Clause 47. The system of clause 43, wherein the one or more processors are further caused to perform a corrective action when compliance is not determined.

Clause 48. The system of clause 43 wherein the one or more processors are further caused to determine a percent of the plurality of slopes that is positive, compare the percent of the plurality of slopes that is positive to a positive slope threshold, and when the percent of the plurality of slopes that is positive is below the positive slope threshold, continue titration of the insulin dose.

Clause 49. The system of clause 48, wherein when the percent of the plurality of slopes that is positive exceeds the positive slope threshold, the one or more processors are further caused to determine a risk of sudden compliance.

Clause 50. The system of clause 49, wherein the one or more processors are further caused to continue titration of the insulin dose when the risk of sudden compliance is determined to be low.

Clause 51. The system of clause 50, wherein the one or more processors are further caused to stop titration of the insulin dose when the risk of sudden compliance is not determined to be low.

Clause 52. The system of any of clauses 49 to 51, wherein the risk of sudden compliance is determined to be low when

$${D}_{next} < \frac{T G {L}_{MIN} - K}{{β}_{max}} + {D}_{NC}$$

wherein Dₙₑₓₜ is a recommended dose, TGL_{MIN} is a minimum glucose metric, K is a predetermined value, βₘₐₓ is a maximum insulin sensitivity factor, and D_{NC} is a dose most recently acknowledged by the user.

Clause 53. A method for titrating an insulin dose, the method comprising:
outputting, by a processor, a dose recommendation for a basal insulin dose on a display;
determining, by the processor, an expected glucose metric based on the dose recommendation;
determining, by the processor, an actual glucose metric based on glucose data received from a glucose monitoring device after the dose recommendation is output;
determining, by the processor, non-compliance with the dose recommendation when the actual glucose metric is not within a predetermined amount of the expected glucose metric; and
eschewing titration of the basal insulin dose by the processor when non-compliance is determined.

Clause 54. The method of clause 53, further comprising determining compliance with the dose recommendation when the actual glucose metric is within the predetermined amount of the expected glucose metric.

Clause 55. The method of clause 53 or 54, wherein the actual glucose metric comprises a titration glucose level.

Clause 56. The method of any of clauses 53 to 55, wherein the expected glucose metric is determined based on an insulin sensitivity factor of the user.

Clause 57. The method of clause 56, further comprising:
determining a titration glucose level corresponding to each of a plurality of doses administered by the user; and
determining the insulin sensitivity factor based on a slope of a fit line for the plurality of titration glucose levels and the plurality of doses administered by the user.

Clause 58. The method of any of clauses 53 to 57, wherein the predetermined amount of the expected glucose metric is based on a variability in the titration glucose level.

Clause 59. The method of any of clauses 53 to 58, further comprising outputting, by the processor, a notification indicating that titration of the basal insulin dose is disabled.

1-22. (canceled)

23. A method for detecting compliance with insulin dose titration recommendations provided by a dose guidance system, the method comprising:
outputting, on a display of the dose guidance system, a new dose recommendation for an insulin dose;
receiving, by a processor in communication with the display, user confirmation of the new dose recommendation;
receiving, by the processor, an indication that a dose was administered within a predetermined period of time after output of the new dose recommendation; and
titrating, by the processor, the insulin dose when user confirmation is received and when the indication that the dose was administered within the predetermined period of time is received; and
eschewing titration of the insulin dose when the user confirmation is not received or when the indication that the dose was administered is not received within the predetermined period of time after output of the new dose recommendation.

24. The method of claim 23, wherein receiving user confirmation of the new dose recommendation comprises receiving a user input to acknowledge the user viewed the new dose recommendation.

25. The method of claim 23, wherein receiving the indication comprises detecting administration of the dose by a dose capture device or by a medication delivery device of the dose guidance system.

26. The method of claim 23, further comprising initiating a corrective action when the user confirmation is not received or when the indication that the dose was administered is not received within the predetermined period of time after output of the new dose recommendation.

27. The method of claim 26, wherein the corrective action comprises outputting a prompt for the user to enter a most recently administered dose amount or outputting a prompt for a user to confirm administration of the new dose recommendation.

28. The method of claim 27, wherein the method further comprises resuming titration of the insulin dose when the most recently administered dose amount entered by the user is the same as the new dose recommendation or when the user confirms administration of the new dose recommendation.

29. The method of claim 26, wherein the corrective action comprises outputting educational information.

30. The method of claim 23, wherein titrating the insulin dose comprises outputting a second new dose recommendation on the display of the dose guidance system.

31. A method for detecting compliance with insulin dose recommendations provided by a dose guidance system, the method comprising:
outputting a first dose recommendation for an insulin dose on a display of the dose guidance system;
determining, by a processor of the dose guidance system, a first glucose metric based on glucose data received from a glucose monitoring device after output of the first dose recommendation;
outputting, by the processor, a second dose recommendation for the insulin dose on the display of the dose guidance system based on the glucose data received after output of the first dose recommendation;
determining, by the processor, a second glucose metric based on the glucose data received after output of the second dose recommendation;
comparing, by the processor, the first glucose metric to the second glucose metric;
determining, by the processor, compliance or non-compliance based on the comparison of the first glucose metric to the second glucose metric;
eschewing titration of the insulin dose by the processor when compliance is not determined; and
outputting a notification on the display indicating that further titration of the insulin dose is disabled.

32. The method of claim 31, wherein the insulin dose is a basal insulin dose.

33. The method of claim 31, wherein the first glucose metric is a titration glucose level.

34. The method of claim 31, wherein the second dose recommendation is greater than the first dose recommendation, and wherein determining compliance or non-compliance comprises determining that the second glucose metric is lower than the first glucose metric.

35. The method of claim 31, further comprising titrating the insulin dose when compliance is determined.

36.-45. (canceled)

46. A method for titrating an insulin dose, the method comprising:
outputting, by a processor, a dose recommendation for a basal insulin dose on a display;
determining, by the processor, an expected glucose metric based on the dose recommendation;
determining, by the processor, an actual glucose metric based on glucose data received from a glucose monitoring device after the dose recommendation is output;
determining, by the processor, non-compliance with the dose recommendation when the actual glucose metric is not within a predetermined amount of the expected glucose metric; and
eschewing titration of the basal insulin dose by the processor when non-compliance is determined.

47. The method of claim 46, further comprising determining compliance with the dose recommendation when the actual glucose metric is within the predetermined amount of the expected glucose metric.

48. The method of claim 46, wherein the actual glucose metric comprises a titration glucose level.

49. The method of claim 46, wherein the expected glucose metric is determined based on an insulin sensitivity factor of the user.

50. The method of claim 49, further comprising:
determining a titration glucose level corresponding to each of a plurality of doses administered by the user; and
determining the insulin sensitivity factor based on a slope of a fit line for the plurality of titration glucose levels and the plurality of doses administered by the user.

51. The method of claim 46, wherein the predetermined amount of the expected glucose metric is based on a variability in the titration glucose level.

52. The method of claim 46, further comprising outputting, by the processor, a notification indicating that titration of the basal insulin dose is disabled.
